SEQUENCE LISTING (7 sequences)

SEQ ID NO: 1 - protein, 149 aa, Mus musculus
APTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 2 - protein, 133 aa, Homo sapiens [sequence omitted]

SEQ ID NO: 3 - protein, 190 aa, Mus musculus
MGAMAPRTLLLLLAAALAPTQTRAGPGSCDLPHTYNLGNKRALTVLEEMRRLPPLSCLKDRKDFGFPLEKVDNQQIQKAQAILVLRDLTQQILNLFTSKDLSATWNATLLDSFCNDLHQQLNDLKACVMQEPPLTQEDSLLAVRTYFHRITVYLRKKKHSLCAWEVIRAEVWRALSSSTNLLARLSEEKE

SEQ ID NO: 4 - protein, 608 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 5 - protein, 609 aa, Homo sapiens [sequence omitted]

SEQ ID NO: 6 - protein, 767 aa, Artificial Sequence
  Extended-PK IL2
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALAGGSGGGGSGGAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 7 - protein, 752 aa, Artificial Sequence
  Extended-PK IL2
MKWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGSGGGGSGGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

(19) United States
(12) Patent Application Publication
(10) Pub. No.: US 2022/0143144 A1
(43) Pub. Date: May 12, 2022

(54) TREATMENT INVOLVING INTERLEUKIN-2 (IL2) AND INTERFERON (IFN)

(71) Applicants: BIONTECH SE, Mainz (DE); TRON-Translationale Onkologie an der Universitätsmedizin der Johannes Gutenberg-Universität Mainz, Mainz (DE)

(72) Inventors: Ugur SAHIN, Mainz (DE); Mathias VORMEHR, Mainz (DE); Lena KRANZ, Mainz (DE); Sina FELLERMEIER-KOPF, Mainz (DE); Alexander MUIK, Mainz (DE); Daniel REIDENBACH, Mainz (DE); Mustafa DIKEN, Mainz (DE); Sebastian KREITER, Mainz (DE)

(21) Appl. No.: 17/442,568

(22) PCT Filed: Apr. 2, 2020
(86) PCT No.: PCT/EP2020/059445

§ 371 Date: Sep. 23, 2021

(30) Foreign Application Priority Data
Apr. 5, 2019 (EP) PCT/EP2019/058707

(51) Int. Cl. A61K 38/20; A61K 38/21; A61P 37/04; A61P 35/00

(52) U.S. Cl. CPC A61K 38/2013; A61P 35/00; A61P 37/04; A61K 38/212

(57) ABSTRACT

The present disclosure relates to methods and agents for enhancing the effect of immune effector cells, in particular immune effector cells that respond to interleukin-2 (IL2), for example effector T cells such as CD8+ T cells. Specifically, the present disclosure relates to methods comprising administering to a subject a polypeptide comprising IL2 or a functional variant thereof or a polynucleotide encoding a polypeptide comprising IL2 or a functional variant thereof and a polypeptide comprising type I interferon (IFN) or a functional variant thereof or a polynucleotide encoding a polypeptide comprising type I interferon or a functional variant thereof.

### TECHNICAL FIELD

The present disclosure relates to methods and agents for enhancing the effect of immune effector cells, in particular immune effector cells that respond to interleukin-2 (IL2), for example effector T cells such as CD8+ T cells. These methods and agents are, in particular, useful for the treatment of diseases characterized by diseased cells expressing an antigen the immune effector cells are directed to. Specifically, the present disclosure relates to methods comprising administering to a subject a polypeptide comprising IL2 or a functional variant thereof (referred to herein generally as “IL2”) or a polynucleotide encoding a polypeptide comprising IL2 or a functional variant thereof and a polypeptide comprising type I interferon (IFN) or a functional variant thereof (referred to herein generally as “interferon” or “IFN”) or a polynucleotide encoding a polypeptide comprising type I interferon or afunctional variant thereof. The IL2 provided to the subject acts on immune effector cells such as effector T cells and results in an enhanced action of the immune effector cells, e.g., by promoting expansion of the immune effector cells, while the interferon prevents or reduces IL2-mediated expansion of regulatory T cells (Treg), which would counteract the action of the immune effector cells. The methods of the disclosure may further comprise administering to the subject vaccine antigen or a polynucleotide coding therefor to provide (optionally following expression of the polynucleotide by appropriate target cells) antigen for stimulation, priming and/or expansion of the immune effector cells. In one embodiment, the immune effector cells carry an antigen receptor such as T cell receptor (TCR) or chimeric antigen receptor (CAR) having a binding specificity for the antigen or a procession product thereof. In one embodiment, the immune effector cells are genetically modified to express the antigen receptor. Alternatively or additionally, the immune effector cells are genetically modified to express an IL2 receptor (IL2R). Such genetic modification may be effected ex vivo or in vitro and subsequently the immune effector cells may be administered to a subject in need of treatment and/or may be effected in vivo in a subject in need of treatment. The immune effector cells may be from the subject in need of treatment and may be endogenous in the subject in need of treatment. The antigen receptor of the immune effector cells may target antigen which is associated with a disease. In one particularly preferred embodiment, the polynucleotide encoding IL2, polynucleotide encoding IFN and/or polynucleotide encoding vaccine antigen administered according to the present disclosure is RNA.

### BACKGROUND

The immune system plays an important role in cancer, autoimmunity, allergy as well as in pathogen-associated diseases. T cells and NK cells are important mediators of anti-tumor immune responses. CD8+ T cells and NK cells can directly lyse tumor cells. CD4+ T cells, on the other hand, can mediate the influx of different immune subsets including CD8+ T cells and NK cells into the tumor. CD4+ T cells are able to license dendritic cells (DCs) for the priming of anti-tumor CD8+ T cell responses and can act directly on tumor cells via IFNγ mediated MHC upregulation and growth inhibition. CD8+ as well as CD4+ tumor specific T cell responses can be induced via vaccination or by adoptive transfer of T cells. In the context of an mRNA based vaccine platform, mRNA may be delivered via liposomal formulation (RNA-lipoplexes, RNA-LPX) into antigen-presenting cells located in secondary lymphoid organs without requirement for any additional adjuvant for immune stimulation (Kreiter, S. et al. Nature 520, 692-696 (2015); Kranz, L. M. et al. Nature 534, 396-401 (2016)).

One potential way of further improving clinical efficacy of T cells is the support and modulation of said cells via cytokines which affect cell survival and function. For example, interleukin-2 (IL2) is a potent immune stimulator, activating diverse cells of the immune system. IL2 is known to support the differentiation, proliferation, survival and effector functions of T cells and NK cells (Blattman, J. NNat. Med. 9, 540-7 (2003)) and has been used for decades in the treatment of late stage malignant melanoma (Maas, R. A., Dullens, H. F. & Den Otter, W. Cancer Immunother. 36, 141-8 (1993)).

However, there are several difficulties connected with the administration of cytokines. Recombinant cytokines have a very short plasma half-life creating the necessity to frequently inject high amounts of cytokine. In case of IL2 this leads to severe side effects such as vascular leak syndrome (VLS) (Rosenberg, S. N. Engl. J. Med. 316, 889-97 (1987)). Furthermore, cytokine administration might cause unwanted effects on immune cells. For example, IL2 is known for its ability to stimulate regulatory T cells (Tregs) more potently than effector T cells (Todd, J. PLoS Med. 13, e1002139 (2016)), as the high-affinity IL2 receptor (IL2Rαβγ) consisting of CD25 (IL2Rα), CD122 (IL2Rβ) and CD132 (IL2Rγ) is expressed on Tregs as well as activated CD4⁺ and CD8⁺ T cells, while the intermediate-affinity receptor (IL2Rβγ), which lacks CD25, is prevalent on naïve and memory T cells as well as NK cells. Tregs are correlated with reduced survival of cancer patients as they can suppress the function of anti-tumor effector T cells and NK cells (Nishikawa, H. & Sakaguchi. Curr. Opin 27, 1-7 (2014)). Attempts to alter IL2 in such a way that it loses preference for CD25 expressing cells, thereby relatively increasing the stimulatory potential on naïve and memory T cells as well as NK cells was shown to improve its anti-tumoral potential (Arenas-Ramirez, N. et al. Sci. Transl. Med. 8, 1-13 (2016)).

Clearly, there is a need for novel strategies to increase the effectiveness of immunotherapies, in particular vaccines such as cancer vaccines, and/or cell-based immunotherapies such as cell-based cancer immunotherapies, including adoptive transfer of (naïve or T cell receptor transgenic or chimeric antigen receptor transgenic) T and NK cells.

In order to address the limitations occurring with cytokine therapy, we herein provide novel strategies to increase the effectiveness of immunotherapies involving IL2 treatment.

The present disclosure provides means and methods for preventing IL2-mediated Treg expansion while retaining beneficial effects of IL2 treatment such as expansion of antigen specific T-cell responses.

We demonstrate that mRNA encoding IL2 fused to serum albumin for prolonged systemic availability (Alb-IL2) significantly activates CD8+ T cells and strongly expands vaccine induced antigen specific T cells in vivo in mice. Alb-IL2 at the same time resulted in massive expansion of Tregs strongly limiting the subsequent expansion of antigen specific T cells. Simultaneous administration of IFNα encoding mRNA together with Alb-IL2 prevented Treg expansion and allowed sustained boosting of vaccine induced antigen specific T cells. In vitro studies with isolated human T cells confirmed that IFNα prevents IL2 mediated activation of Tregs without strongly affecting CD8+ T cell activation.

### SUMMARY

The present invention generally embraces the immunotherapeutic treatment of a subject comprising the administration of a polypeptide comprising IL2 or afunctional variant thereof or a polynucleotide encoding a polypeptide comprising IL2 or a functional variant thereof in order to increase the effectiveness of the immunotherapies and the administration of a polypeptide comprising type I interferon or a functional variant thereof or a polynucleotide encoding a polypeptide comprising type I interferon or a functional variant thereof in order to reduce or prevent IL2-mediated unwanted effects, in particular Treg expansion. The immunotherapies may comprise vaccine therapies and/or cell-based cancer immunotherapies such as TIL- or T cell-based treatments, for example TCR- or CAR-transgenic T cell-based treatments using, for example autologous cells. In general, immune effector cells that are stimulated using the treatments described herein may target cells expressing an antigen such as diseased cells, in particular cancer cells expressing a tumor antigen. The target cells may express the antigen on the cell surface or may present a procession product of the antigen. In one embodiment, the antigen is a tumor-associated antigen and the disease is cancer. Such treatment provides for the selective eradication of cells that express an antigen, thereby minimizing adverse effects to normal cells not expressing the antigen. The immune effector cells (optionally genetically modified to express an antigen receptor) that are to be stimulated by IL2 administration and which preferably have an endogenous IL2 receptor (IL2R) (or are optionally genetically modified to express an IL2R) are targeted to the antigen or a procession product thereof and thus, to a target cell population or target tissue expressing the antigen. Such immune effector cells may be administered to a subject in need of treatment or may be endogenous to a subject in need of treatment. In one embodiment, the immune effector cells carry an IL2 receptor (IL2R). In one embodiment, the immune effector cells are genetically modified to express an IL2R. In one embodiment, the immune effector cells carry an antigen receptor such as T cell receptor (TCR) or chimeric antigen receptor (CAR) having a binding specificity for the target antigen or a procession product thereof. In one embodiment, the immune effector cells are genetically modified to express the antigen receptor. Such genetic modification to express an IL2R and/or antigen receptor may be effected ex vivo or in vitro and subsequently the immune effector cells may be administered to a subject in need of treatment or may be effected in vivo in a subject in need of treatment, or may be effected by a combination of ex vivo or in vitro and in vivo modification. In one embodiment, vaccine antigen or polynucleotide coding therefor is administered to provide (optionally following expression of the polynucleotide by appropriate target cells) antigen for stimulation, priming and/or expansion of the immune effector cells, which are targeted to target antigen or a procession product thereof. In one embodiment, the immune response which is to be induced according to the present disclosure is an immune response to a target cell population or target tissue expressing an antigen the immune effector cells are directed to. In one embodiment, the immune response which is to be induced according to the present disclosure is a T cell-mediated immune response. In one embodiment, the immune response is an anti-tumor immune response and the target cell population or target tissue is tumor cells or tumor tissue.

The methods and agents described herein are particularly effective if IL2 is attached to a pharmacokinetic modifying group (hereafter referred to as “extended-pharmacokinetic (PK) IL2”). The methods and agents described herein are particularly effective if the polynucleotide encoding IL2 such as extended-PK IL2 is RNA and/or the polynucleotide encoding a type I interferon is RNA. In one embodiment, said RNA is targeted to the liver for systemic availability. Liver cells can be efficiently transfected and are able to produce large amounts of protein. Vaccine antigen-encoding RNA is preferably targeted to secondary lymphoid organs.

In one aspect, provided herein is a method for inducing an immune response in a subject comprising administering to the subject:

a. a polypeptide comprising IL2 or a functional variant thereof or a polynucleotide encoding a polypeptide comprising IL2 or a functional variant thereof; and
b. a polypeptide comprising type I interferon or a functional variant thereof or a polynucleotide encoding a polypeptide comprising type I interferon or a functional variant thereof.

In one embodiment, the method further comprises administering to the subject:

c. a peptide or protein comprising an epitope for inducing an immune response against an antigen in the subject or a polynucleotide encoding the peptide or protein.

In one embodiment, the polynucleotide encoding a polypeptide comprising IL2 or a functional variant thereof is RNA, the polynucleotide encoding a polypeptide comprising type I interferon or a functional variant thereof is RNA and optionally the polynucleotide encoding the peptide or protein is RNA.

In one aspect, provided herein is a method for inducing an immune response in a subject comprising administering to the subject:

a. RNA encoding a polypeptide comprising IL2 or afunctional variant thereof; and
b. RNA encoding a polypeptide comprising type I interferon or a functional variant thereof.

In one embodiment, the method further comprises administering to the subject:

c. RNA encoding a peptide or protein comprising an epitope for inducing an immune response against an antigen in the subject.

In one embodiment, the immune response is a T cell-mediated immune response.

In one embodiment, the subject has a disease, disorder or condition associated with expression or elevated expression of an antigen.

In one aspect, provided herein is a method for treating a subject having a disease, disorder or condition associated with expression or elevated expression of an antigen comprising administering to the subject:

a. a polypeptide comprising IL2 or a functional variant thereof or a polynucleotide encoding a polypeptide comprising IL2 or a functional variant thereof;
b. a polypeptide comprising type I interferon or a functional variant thereof or a polynucleotide encoding a polypeptide comprising type I interferon or a functional variant thereof; and
c. a peptide or protein comprising an epitope for inducing an immune response against the antigen in the subject or a polynucleotide encoding the peptide or protein.

In one embodiment, the polynucleotide encoding a polypeptide comprising IL2 or a functional variant thereof is RNA, the polynucleotide encoding a polypeptide comprising type I interferon or a functional variant thereof is RNA and the polynucleotide encoding the peptide or protein is RNA.

In one aspect, provided herein is a method for treating a subject having a disease, disorder or condition associated with expression or elevated expression of an antigen comprising administering to the subject:

a. RNA encoding a polypeptide comprising IL2 or a functional variant thereof;
b. RNA encoding a polypeptide comprising type I interferon or a functional variant thereof; and
c. RNA encoding a peptide or protein comprising an epitope for inducing an immune response against the antigen in the subject.

In one embodiment, the disease, disorder or condition is cancer and the antigen is a tumor-associated antigen.

In one embodiment, the polypeptide comprising IL2 or a functional variant thereof is extended pharmacokinetic (PK) IL2. In one embodiment, the extended-PK IL2 comprises a fusion protein. In one embodiment, the fusion protein comprises a moiety of IL2 or a functional variant thereof and a moiety selected from the group consisting of serum albumin, an immunoglobulin fragment, transferrin, Fn3, and variants thereof. In one embodiment, the serum albumin comprises mouse serum albumin or human serum albumin. In one embodiment, the immunoglobulin fragment comprises an immunoglobulin Fc domain.

In one embodiment, the method of any aspect is a method for treating or preventing cancer in a subject, optionally wherein the antigen is a tumor-associated antigen.

In one embodiment, administration of a polypeptide comprising IL2 or a functional variant thereof or a polynucleotide, in particular RNA, encoding a polypeptide comprising IL2 or a functional variant thereof and optionally a peptide or protein comprising an epitope for inducing an immune response against an antigen in the subject or a polynucleotide, in particular RNA, encoding the peptide or protein, induces antigen-specific T cells.

In one embodiment, administration of a polypeptide comprising type I interferon or a functional variant thereof or a polynucleotide, in particular RNA, encoding a polypeptide comprising type I interferon or a functional variant thereof reduces the number of regulatory T cells.

In one embodiment, administration of a polypeptide comprising type I interferon or a functional variant thereof or a polynucleotide, in particular RNA, encoding a polypeptide comprising type I interferon or a functional variant thereof reduces IL2 mediated expansion of regulatory T cells.

In one embodiment, administration of a polypeptide comprising type I interferon or a functional variant thereof or a polynucleotide, in particular RNA, encoding a polypeptide comprising type I interferon or a functional variant thereof increases the ratio of antigen-specific T cells to T regulatory cells.

In one embodiment of all aspects, the type I interferon is interferon-α.

In one aspect, provided herein is a medical preparation comprising:

a. a polypeptide comprising IL2 or a functional variant thereof or a polynucleotide encoding a polypeptide comprising IL2 or a functional variant thereof; and
b. a polypeptide comprising type I interferon or a functional variant thereof or a polynucleotide encoding a polypeptide comprising type I interferon or a functional variant thereof.

In one embodiment, the medical preparation further comprises:

c. a peptide or protein comprising an epitope for inducing an immune response against an antigen in a subject or a polynucleotide encoding the peptide or protein.

In one embodiment, the polynucleotide encoding a polypeptide comprising IL2 or a functional variant thereof is RNA, the polynucleotide encoding a polypeptide comprising type I interferon or a functional variant thereof is RNA and optionally the polynucleotide encoding the peptide or protein is RNA.

In one embodiment, the medical preparation is a kit.

In one embodiment, the medical preparation comprises the polypeptide comprising IL2 or a functional variant thereof or polynucleotide encoding a polypeptide comprising IL2 or a functional variant thereof, the polypeptide comprising type I interferon or a functional variant thereof or polynucleotide encoding a polypeptide comprising type I interferon or afunctional variant thereof and optionally the peptide or protein or polynucleotide encoding the peptide or protein in separate containers.

In one embodiment, the medical preparation further comprises instructions for use of the medical preparation for treating or preventing cancer, optionally wherein the antigen is a tumor-associated antigen.

In one embodiment, the medical preparation is a pharmaceutical composition.

In one embodiment, the pharmaceutical composition further comprises one or more pharmaceutically acceptable carriers, diluents and/or excipients.

In one aspect, provided herein is a medical preparation comprising:

a. RNA encoding a polypeptide comprising IL2 or a functional variant thereof; and
b. RNA encoding a polypeptide comprising type I interferon or a functional variant thereof.

In one embodiment, the medical preparation further comprises:

c. RNA encoding a peptide or protein comprising an epitope for inducing an immune response against an antigen in a subject.

In one embodiment, the medical preparation is a kit.

In one embodiment, the medical preparation comprises the RNA encoding a polypeptide comprising IL2 or afunctional variant thereof, the RNA encoding a polypeptide comprising type I interferon or afunctional variant thereof and optionally the RNA encoding a peptide or protein in separate containers.

In one embodiment, the medical preparation further comprises instructions for use of the medical preparation for treating or preventing cancer, optionally wherein the antigen is a tumor-associated antigen.

In one embodiment, the medical preparation is a pharmaceutical composition.

In one embodiment, the pharmaceutical composition further comprises one or more pharmaceutically acceptable carriers, diluents and/or excipients.

In one embodiment of the medical preparation, the immune response is a T cell-mediated immune response.

In one embodiment, the polypeptide comprising IL2 or a functional variant thereof is extended pharmacokinetic (PK) IL2. In one embodiment, the extended-PK IL2 comprises a fusion protein. In one embodiment, the fusion protein comprises a moiety of IL2 or a functional variant thereof and a moiety selected from the group consisting of serum albumin, an immunoglobulin fragment, transferrin, Fn3, and variants thereof. In one embodiment, the serum albumin comprises mouse serum albumin or human serum albumin. In one embodiment, the immunoglobulin fragment comprises an immunoglobulin Fc domain.

In one aspect, provided herein is the medical preparation described herein for pharmaceutical use.

In one embodiment, the pharmaceutical use comprises a therapeutic or prophylactic treatment of a disease or disorder.

In one aspect, provided herein is the medical preparation described herein for use in a method for treating or preventing cancer in a subject, optionally wherein the antigen is a tumor-associated antigen.

In one embodiment of all aspects, the type I interferon is interferon-α.

### BRIEF DESCRIPTION OF THE DRAWINGS

FIG. 1: Combined treatment of mAlb-mIL2 and T-cell vaccination results in a temporary boost of ovalbumin specific CD8+ T cells

A, Outline of the experiment. B-D, Number of Ovalbumin (OVA) specific CD8+ T cells on day 7 (B), 14 (C), and 21(D) in peripheral blood. E, Frequency of OVA specific T cells over time. F, Frequency of CD4+CD25+FoxP3+ Tregs among CD4+ T cells over time. For statistical analysis a two-tailed unpaired student's t-test (B-D) or two-way analysis of variance and Sidak's multiple comparison test (E,F) was applied. ns; P>0.05, *: P≤0.05, **: P≤0.01, ***; P≤0.001, ***; P≤0.0001. Mean±s.e.m. is shown.

FIG. 2: Combined treatment of mAlb-mIL2 and T-cell vaccination results In a temporary boost of gp70 specific CD8+ T cells

A, Outline of the experiment. B-D, Number of gp70 specific CD8+ T cells on day 7 (B), 14 (C), and 21 (D) in peripheral blood. E, Frequency of gp70 specific T cells over time. F, Frequency of CD4+CD25+FoxP3+ Tregs among CD4+ T cells over time. For statistical analysis a two-tailed unpaired students t-test (B-D) or two-way analysis of variance and Sidak's multiple comparison test (E,F) was applied. ns; P>0.05, *: P≤0.05, **: P≤0.01, ***; P≤0.001, ***; P≤0.0001. Mean±s.e.m. is shown.

FIG. 3: IFNα limits IL2 mediated Treg expansion resulting In robust priming of OVA specific T cells In vivo

A, Outline of the experiment. B, D, Frequency of OVA specific CD8+ T cells on day 7 (B) and 14 (D) in peripheral blood. C, Frequency of CD4+CD25+FoxP3+ Tregs among CD4+ T cells on day 7. For statistical analysis a one-way analysis of variance followed by Sidak's multiple comparison test was applied. ns; P>0.05, *: P≤0.05, **: P≤0.01, ***; P≤0.001, ***; P≤0.0001. Mean±s.e.m. is shown.

FIG. 4: IFNα limits IL2 mediated Treg expansion resulting in robust priming of gp70 specific T cells In vivo

A, Outline of the experiment. B, D, Number of gp70 specific CD8+ T cells on day 7 (B) and 21 (D) in peripheral blood. C, Frequency of CD4+CD25+FoxP3+ Tregs among CD4+ T cells on day 7. For statistical analysis a one-way analysis of variance followed by Sidak's multiple comparison test was applied. ns; P>0.05, *: P≤0.05, **: P≤0.01, ***; P≤0.001, ***; P≤0.0001. Mean±s.e.m. is shown.

FIG. 5: IFNα limits IL2 mediated Treg expansion but not CD8+ T cell expansion In vitro

CellTrace FarRed-labeled isolated Tregs (CD4+CD25+) were co-cultured at a 1:1 ratio with autologous CFSE-labeled PBMCs in the presence of a sub-optimal concentration of anti-CD3 antibody (clone UCHT1) and treated with 5% hAlb-hIL2-containing supernatant. Co-cultures were incubated either with 10,000 U/mL hIFNα2b, 625 U/mL hIFNα2b or kept without hIFNα2b. Proliferation of CD4+CD25+ Tregs and CD8+ T cells was measured by flow cytometry after 4 days of incubation. Data is shown from two different PBMC donors (A, B) as mean values of expansion indices as calculated using FlowJo v10.5 software. Error bars indicate the variation within the experiment (two replicates).

FIG. 6: IFNα and IL2 combination therapy leads to a synergistic anti-tumoral effect in mice. A, Treatment regimen and analysis schedule. B, Median tumor size per group. For statistical analysis, a two-way ANOVA followed by Dunnett's test was performed comparing all groups to the mAlb control. C, Tumor growth curves of single mice. Dotted vertical lines indicate treatments. D, Survival of mice. For statistical comparison of survival between the IL2 and IFNα combination group and the IL2 monotherapy group, a log-rank test was performed. E, Fraction of CD8⁺ T cells among CD45⁺ cells at day 29 (left) and day 35 (right). Mean (line) and individual values (symbols) are shown. Dotted horizontal lines indicate the mean of the mAlb group. One-way ANOVA following Tukey's test was performed to identify significant differences. ns; P>0.05, *: P≤0.05, **: P≤0.01, ***; P≤0.001, ***; P≤0.0001.

### DETAILED DESCRIPTION

Although the present disclosure is described in detail below, it is to be understood that this disclosure is not limited to the particular methodologies, protocols and reagents described herein as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, H. G. W. Leuenberger, B. Nagel, and H. Kölbl, Eds., Helvetica Chimica Acta, CH-4010 Basel, Switzerland, (1995).

The practice of the present disclosure will employ, unless otherwise indicated, conventional methods of chemistry, biochemistry, cell biology, immunology, and recombinant DNA techniques which are explained in the literature in the field (cf., e.g., Molecular Cloning: A Laboratory Manual, 2nd Edition, J. Sambrook et al. eds., Cold Spring Harbor Laboratory Press, Cold Spring Harbor 1989).

In the following, the elements of the present disclosure will be described. These elements are listed with specific embodiments, however, it should be understood that they may be combined in any manner and in any number to create additional embodiments. The variously described examples and embodiments should not be construed to limit the present disclosure to only the explicitly described embodiments. This description should be understood to disclose and encompass embodiments which combine the explicitly described embodiments with any number of the disclosed elements. Furthermore, any permutations and combinations of all described elements should be considered disclosed by this description unless the context indicates otherwise.

The term “about” means approximately or nearly, and in the context of a numerical value or range set forth herein in one embodiment means±20%, ±10%, ±5%, or ±3% of the numerical value or range recited or claimed.

The terms “a” and “an” and “the” and similar reference used in the context of describing the disclosure (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it was individually recited herein. All methods described herein can be performed in any suitable order unless otherwise indicated herein or otherwise clearly contradicted by context. The use of any and all examples, or exemplary language (e.g., “such as”), provided herein is intended merely to better illustrate the disclosure and does not pose a limitation on the scope of the claims. No language in the specification should be construed as indicating any non-claimed element essential to the practice of the disclosure.

Unless expressly specified otherwise, the term “comprising” is used in the context of the present document to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the present disclosure was not entitled to antedate such disclosure.

In the following, definitions will be provided which apply to all aspects of the present disclosure. The following terms have the following meanings unless otherwise indicated. Any undefined terms have their art recognized meanings.

### Definitions

Terms such as “reduce”, “decrease”, “inhibit” or “impair” as used herein relate to an overall decrease or the ability to cause an overall decrease, preferably of 5% or greater, 10% or greater, 20% or greater, more preferably of 50% or greater, and most preferably of 75% or greater, in the level, e.g. in the level of binding. Terms such as “increase”, “enhance” or “exceed” preferably relate to an increase or enhancement by about at least 10%, preferably at least 20%, preferably at least 30%, more preferably at least 40%, more preferably at least 50%, even more preferably at least 80%, and most preferably at least 100%, at least 200%, at least 500%, or even more.

According to the disclosure, the term “peptide” refers to substances which comprise about two or more, about 3 or more, about 4 or more, about 6 or more, about 8 or more, about 10 or more, about 13 or more, about 16 or more, about 20 or more, and up to about 50, about 100 or about 150, consecutive amino acids linked to one another via peptide bonds. The term “protein” or “polypeptide” refers to large peptides, in particular peptides having at least about 150 amino acids, but the terms “peptide”, “protein” and “polypeptide” are used herein usually as synonyms.

A “therapeutic protein” has a positive or advantageous effect on a condition or disease state of a subject when provided to the subject in a therapeutically effective amount. In one embodiment, a therapeutic protein has curative or palliative properties and may be administered to ameliorate, relieve, alleviate, reverse, delay onset of or lessen the severity of one or more symptoms of a disease or disorder. A therapeutic protein may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Examples of therapeutically active proteins include, but are not limited to, cytokines, and antigens for vaccination.

“Fragment”, with reference to an amino acid sequence (peptide or protein), relates to a part of an amino acid sequence, i.e. a sequence which represents the amino acid sequence shortened at the N-terminus and/or C-terminus. A fragment shortened at the C-terminus (N-terminal fragment) is obtainable e.g. by translation of a truncated open reading frame that lacks the 3′-end of the open reading frame. A fragment shortened at the N-terminus (C-terminal fragment) is obtainable e.g. by translation of a truncated open reading frame that lacks the 5′-end of the open reading frame, as long as the truncated open reading frame comprises a start codon that serves to initiate translation. A fragment of an amino acid sequence comprises e.g. at least 50%, at least 60%, at least 70%, at least 80%, at least 90% of the amino acid residues from an amino acid sequence. A fragment of an amino acid sequence preferably comprises at least 6, in particular at least 8, at least 12, at least 15, at least 20, at least 30, at least 50, or at least 100 consecutive amino acids from an amino acid sequence.

By “variant” or “variant protein” or “variant polypeptide” herein is meant a protein that differs from a wild type protein by virtue of at least one amino acid modification. The parent polypeptide may be a naturally occurring or wild type (WT) polypeptide, or may be a modified version of a wild type polypeptide. Preferably, the variant polypeptide has at least one amino acid modification compared to the parent polypeptide, e.g. from 1 to about 20 amino acid modifications, and preferably from 1 to about 10 or from 1 to about 5 amino acid modifications compared to the parent.

By “parent polypeptide”, “parent protein”, “precursor polypeptide”, or “protein” as used herein is meant an unmodified polypeptide that is subsequently modified to generate a variant. A parent polypeptide may be a wild type polypeptide, or a variant or engineered version of a wild type polypeptide.

By “wild type” or “WT” or “native” herein is meant an amino acid sequence that is found in nature, including allelic variations. A wild type protein or polypeptide has an amino acid sequence that has not been intentionally modified.

For the purposes of the present disclosure, “variants” of an amino acid sequence (peptide, protein or polypeptide) comprise amino acid insertion variants, amino acid addition variants, amino acid deletion variants and/or amino acid substitution variants. The term “variant” includes all splice variants, posttranslationally modified variants, conformations, isoforms and species homologs, in particular those which are naturally expressed by cells. The term “variant” includes, in particular, fragments of an amino acid sequence.

Amino acid insertion variants comprise insertions of single or two or more amino acids in a particular amino acid sequence. In the case of amino acid sequence variants having an insertion, one or more amino acid residues are inserted into a particular site in an amino acid sequence, although random insertion with appropriate screening of the resulting product is also possible. Amino acid addition variants comprise amino- and/or carboxy-terminal fusions of one or more amino acids, such as 1, 2, 3, 5, 10, 20, 30, 50, or more amino acids. Amino acid deletion variants are characterized by the removal of one or more amino acids from the sequence, such as by removal of 1, 2, 3, 5, 10, 20, 30, 50, or more amino acids. The deletions may be in any position of the protein. Amino acid deletion variants that comprise the deletion at the N-terminal and/or C-terminal end of the protein are also called N-terminal and/or C-terminal truncation variants. Amino acid substitution variants are characterized by at least one residue in the sequence being removed and another residue being inserted in its place. Preference is given to the modifications being in positions in the amino acid sequence which are not conserved between homologous proteins or peptides and/or to replacing amino acids with other ones having similar properties. Preferably, amino acid changes in peptide and protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In one embodiment, conservative amino acid substitutions include substitutions within the following groups:

glycine, alanine;
valine, isoleucine, leucine;
aspartic acid, glutamic acid;
asparagine, glutamine;
serine, threonine;
lysine, arginine; and
phenylalanine, tyrosine.

Preferably the degree of similarity, preferably identity between a given amino acid sequence and an amino acid sequence which is a variant of said given amino acid sequence will be at least about 60%, 70%, 80%, 81%, 82%, 83%, 84%, 85%, 86%, 87%, 88%, 89%, 90%, 91%, 92%, 93%, 94%, 95%, 96%, 97%, 98%, or 99%. The degree of similarity or identity is given preferably for an amino acid region which is at least about 10%, at least about 20%, at least about 30%, at least about 40%, at least about 50%, at least about 60%, at least about 70%, at least about 80%, at least about 90% or about 100% of the entire length of the reference amino acid sequence. For example, if the reference amino acid sequence consists of 200 amino acids, the degree of similarity or identity is given preferably for at least about 20, at least about 40, at least about 60, at least about 80, at least about 100, at least about 120, at least about 140, at least about 160, at least about 180, or about 200 amino acids, preferably continuous amino acids. In preferred embodiments, the degree of similarity or identity is given for the entire length of the reference amino acid sequence. The alignment for determining sequence similarity, preferably sequence identity can be done with art known tools, preferably using the best sequence alignment, for example, using Align, using standard settings, preferably EMBOSS::needle, Matrix: Blosum62, Gap Open 10.0, Gap Extend 0.5.

“Sequence similarity” indicates the percentage of amino acids that either are identical or that represent conservative amino acid substitutions. “Sequence identity” between two amino acid sequences indicates the percentage of amino acids that are identical between the sequences.

The term “percentage identity” is intended to denote a percentage of amino acid residues which are identical between the two sequences to be compared, obtained after the best alignment, this percentage being purely statistical and the differences between the two sequences being distributed randomly and over their entire length. Sequence comparisons between two amino acid sequences are conventionally carried out by comparing these sequences after having aligned them optimally, said comparison being carried out by segment or by “window of comparison” in order to identify and compare local regions of sequence similarity. The optimal alignment of the sequences for comparison may be produced, besides manually, by means of the local homology algorithm of Smith and Waterman, 1981, Ads App. Math. 2, 482, by means of the local homology algorithm of Neddleman and Wunsch, 1970, J. Mol. Biol. 48, 443, by means of the similarity search method of Pearson and Lipman, 1988, Proc. Natl Acad. Sci. USA 85, 2444, or by means of computer programs which use these algorithms (GAP, BESTFIT, FASTA, BLAST P, BLAST N and TFASTA in Wisconsin Genetics Software Package, Genetics Computer Group, 575 Science Drive, Madison, Wis.).

The percentage identity is calculated by determining the number of identical positions between the two sequences being compared, dividing this number by the number of positions compared and multiplying the result obtained by 100 so as to obtain the percentage identity between these two sequences.

Homologous amino acid sequences exhibit according to the disclosure at least 40%, in particular at least 50%, at least 60%, at least 70%, at least 80%, at least 90% and preferably at least 95%, at least 98 or at least 99% identity of the amino acid residues.

The amino acid sequence variants described herein may readily be prepared by the skilled person, for example, by recombinant DNA manipulation. The manipulation of DNA sequences for preparing peptides or proteins having substitutions, additions, insertions or deletions, is described in detail in Sambrook et al. (1989), for example. Furthermore, the peptides and amino acid variants described herein may be readily prepared with the aid of known peptide synthesis techniques such as, for example, by solid phase synthesis and similar methods.

In one embodiment, a fragment or variant of an amino acid sequence (peptide or protein) is preferably a “functional fragment” or “functional variant”. The term “functional fragment” or “functional variant” of an amino acid sequence relates to any fragment or variant exhibiting one or more functional properties identical or similar to those of the amino acid sequence from which it is derived, i.e., it is functionally equivalent. With respect to cytokines such as IL2, one particular function is one or more immunomodulatory activities displayed by the amino acid sequence from which the fragment or variant is derived and/or binding to the receptor(s) the amino acid sequence from which the fragment or variant is derived binds to. The term “functional fragment” or “functional variant”, as used herein, in particular refers to a variant molecule or sequence that comprises an amino acid sequence that is altered by one or more amino acids compared to the amino acid sequence of the parent molecule or sequence and that is still capable of fulfilling one or more of the functions of the parent molecule or sequence, e.g., binding to a target molecule or contributing to binding to a target molecule. In one embodiment, the modifications in the amino acid sequence of the parent molecule or sequence do not significantly affect or alter the binding characteristics of the molecule or sequence. In different embodiments, binding of the functional fragment or functional variant may be reduced but still significantly present, e.g., binding of the functional variant may be at least 50%, at least 60%, at least 70%, at least 80%, or at least 90% of the parent molecule or sequence. However, in other embodiments, binding of the functional fragment or functional variant may be enhanced compared to the parent molecule or sequence.

An amino acid sequence (peptide, protein or polypeptide) “derived from” a designated amino acid sequence (peptide, protein or polypeptide) refers to the origin of the first amino acid sequence. Preferably, the amino acid sequence which is derived from a particular amino acid sequence has an amino acid sequence that is identical, essentially identical or homologous to that particular sequence or a fragment thereof. Amino acid sequences derived from a particular amino acid sequence may be variants of that particular sequence or a fragment thereof. For example, it will be understood by one of ordinary skill in the art that the antigens and cytokines (e.g., IL2) suitable for use herein may be altered such that they vary in sequence from the naturally occurring or native sequences from which they were derived, while retaining the desirable activity of the native sequences.

As used herein, an “instructional material” or “instructions” includes a publication, a recording, a diagram, or any other medium of expression which can be used to communicate the usefulness of the compositions and methods of the invention. The instructional material of the kit of the invention may, for example, be affixed to a container which contains the compositions of the invention or be shipped together with a container which contains the compositions. Alternatively, the instructional material may be shipped separately from the container with the intention that the instructional material and the compositions be used cooperatively by the recipient.

“Isolated” means altered or removed from the natural state. For example, a nucleic acid or a peptide naturally present in a living animal is not “isolated”, but the same nucleic acid or peptide partially or completely separated from the coexisting materials of its natural state is “isolated”. An isolated nucleic acid or protein can exist in substantially purified form, or can exist in a non-native environment such as, for example, a host cell.

The term “recombinant” in the context of the present invention means “made through genetic engineering”. Preferably, a “recombinant object” such as a recombinant cell in the context of the present invention is not occurring naturally.

The term “naturally occurring” as used herein refers to the fact that an object can be found in nature. For example, a peptide or nucleic acid that is present in an organism (including viruses) and can be isolated from a source in nature and which has not been intentionally modified by man in the laboratory is naturally occurring.

The term “genetic modification” includes the transfection of cells with nucleic acid. The term “transfection” relates to the introduction of nucleic acids, in particular RNA, into a cell. For purposes of the present invention, the term “transfection” also includes the introduction of a nucleic acid into a cell or the uptake of a nucleic acid by such cell, wherein the cell may be present in a subject, e.g., a patient. Thus, according to the present invention, a cell for transfection of a nucleic acid described herein can be present in vitro or in vivo, e.g. the cell can form part of an organ, a tissue and/or an organism of a patient. According to the invention, transfection can be transient or stable. For some applications of transfection, it is sufficient if the transfected genetic material is only transiently expressed. Since the nucleic acid introduced in the transfection process is usually not integrated into the nuclear genome, the foreign nucleic acid will be diluted through mitosis or degraded. Cells allowing episomal amplification of nucleic acids greatly reduce the rate of dilution. If it is desired that the transfected nucleic acid actually remains in the genome of the cell and its daughter cells, a stable transfection must occur. Such stable transfection can be achieved by using virus-based systems or transposon-based systems for transfection. Generally, cells that are genetically modified to express a receptor polypeptide such as an antigen receptor or IL2 receptor are stably transfected with nucleic acid encoding the receptor, while, generally, nucleic acid encoding a cytokine such as IL2 or type I interferon and/or nucleic acid encoding antigen is transiently transfected into cells. RNA can be transfected into cells to transiently express its coded protein.

Immune Effector Cells

The immune effector cells, in particular IL2 responsive immune effector cells (immune effector cells harboring an IL2R) to be used herein may be administered to a subject in need of treatment or may be endogenously present in a subject in need of treatment. Administering to the subject IL2 or a polynucleotide encoding IL2 allows for the stimulation of the immune effector cells. The methods and agents described herein are, in particular, useful for the treatment of diseases characterized by diseased cells expressing an antigen the immune effector cells are directed to. In one embodiment, the immune effector cells carry an antigen receptor such a T cell receptor (TCR) or chimeric antigen receptor (CAR) having a binding specificity for the antigen or a procession product thereof. In one embodiment, the immune effector cells are present in a subject to be treated and are genetically modified in vivo in the subject to express an antigen receptor. In one embodiment, immune effector cells either from a subject to be treated or from a different subject are administered to the subject to be treated. The administered immune effector cells may be genetically modified ex vivo prior to administration or genetically modified in vivo in the subject following administration to express an antigen receptor. In one embodiment, an antigen receptor is endogenous to the immune effector cells. In one embodiment, the immune effector cells are genetically modified, ex vivo or in vivo, to express an IL2R. Thus, such genetic modification with IL2R may be effected in vitro (optionally together with genetic modification by the antigen receptor) and subsequently the immune effector cells administered to a subject in need of treatment or may be effected in vivo (optionally together with genetic modification by the antigen receptor) in a subject in need of treatment.

Thus, the immune effector cells that are to be stimulated by IL2 include any cell which, either naturally or following transfection with one or more IL2R polypeptides, is responsive to IL2. Such responsiveness includes activation, differentiation, proliferation, survival and/or indication of one or more immune effector functions. The cells include, in particular, cells with lytic potential, in particular lymphoid cells, and are preferably T cells, in particular cytotoxic lymphocytes, preferably selected from cytotoxic T cells, natural killer (NK) cells, and lymphokine-activated killer (LAK) cells. Upon activation, each of these cytotoxic lymphocytes triggers the destruction of target cells. For example, cytotoxic T cells trigger the destruction of target cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target cells. The cells used in connection with the present invention will preferably be autologous cells, although heterologous cells or allogenic cells can be used. In one embodiment, immune effector cells that are to be stimulated by IL2 are endogenous to a subject being treated.

The term “effector functions” in the context of the present invention includes any functions mediated by components of the immune system that result, for example, in the killing of diseased cells such as tumor cells, or in the inhibition of tumor growth and/or inhibition of tumor development, including inhibition of tumor dissemination and metastasis. Preferably, the effector functions in the context of the present invention are T cell mediated effector functions. Such functions comprise in the case of a helper T cell (CD4⁺ T cell) the release of cytokines and/or the activation of CD8⁺ lymphocytes (CTLs) and/or B cells, and in the case of CTL the elimination of cells, i.e., cells characterized by expression of an antigen, for example, via apoptosis or perforin-mediated cell lysis, production of cytokines such as IFN-γ and TNF-α, and specific cytolytic killing of antigen expressing target cells.

The term “immune effector cell” or “immunoreactive cell” in the context of the present invention relates to a cell which exerts effector functions during an immune reaction. An “immune effector cell” in one embodiment is capable of binding an antigen such as an antigen presented in the context of MHC on a cell or expressed on the surface of a cell and mediating an immune response. For example, immune effector cells comprise T cells (cytotoxic T cells, helper T cells, tumor infiltrating T cells), B cells, natural killer cells, neutrophils, macrophages, and dendritic cells. Preferably, in the context of the present invention, “immune effector cells” are T cells, preferably CD4⁺ and/or CD8⁺ T cells. According to the invention, the term “immune effector cell” also includes a cell which can mature into an immune cell (such as T cell, in particular T helper cell, or cytolytic T cell) with suitable stimulation. Immune effector cells comprise CD34⁺ hematopoietic stem cells, immature and mature T cells and immature and mature B cells. The differentiation of T cell precursors into a cytolytic T cell, when exposed to an antigen, is similar to clonal selection of the immune system.

Preferably, an “immune effector cell” recognizes an antigen with some degree of specificity, in particular if presented in the context of MHC or present on the surface of diseased cells such as cancer cells.

Preferably, said recognition enables the cell that recognizes an antigen to be responsive or reactive. If the cell is a helper T cell (CD4⁺ T cell) such responsiveness or reactivity may involve the release of cytokines and/or the activation of CD8⁺ lymphocytes (CTLs) and/or B cells. If the cell is a CTL such responsiveness or reactivity may involve the elimination of cells, i.e., cells characterized by expression of an antigen, for example, via apoptosis or perforin-mediated cell lysis. According to the invention, CTL responsiveness may include sustained caicium flux, cell division, production of cytokines such as IFN-γ and TNF-α, up-regulation of activation markers such as CD44 and CD69, and specific cytolytic killing of antigen expressing target cells. CTL responsiveness may also be determined using an artificial reporter that accurately indicates CTL responsiveness. Such CTL that recognizes an antigen and are responsive or reactive are also termed “antigen-responsive CTL” herein.

In one embodiment, the immune effector cells are CAR-expressing immune effector cells. In one embodiment, the immune effector cells are TCR-expressing immune effector cells.

The immune effector cells to be used according to the invention may express an endogenous antigen receptor such as T cell receptor or B cell receptor or may lack expression of an endogenous antigen receptor.

A “lymphoid cell” is a cell which, optionally after suitable modification, e.g. after transfer of an antigen receptor such as a TCR or a CAR, is capable of producing an immune response such as a cellular immune response, or a precursor cell of such cell, and includes lymphocytes, preferably T lymphocytes, lymphoblasts, and plasma cells. A lymphoid cell may be an immune effector cell as described herein. A preferred lymphoid cell is a T cell which can be modified to express an antigen receptor on the cell surface. In one embodiment, the lymphoid cell lacks endogenous expression of a T cell receptor.

The terms “T cell” and “T lymphocyte” are used interchangeably herein and include T helper cells (CD4+ T cells) and cytotoxic T cells (CTLs, CD8+ T cells) which comprise cytolytic T cells. The term “antigen-specific T cell” or similar terms relate to a T cell which recognizes the antigen to which the T cell is targeted and preferably exerts effector functions of T cells. T cells are considered to be specific for antigen if the cells kill target cells expressing an antigen. T cell specificity may be evaluated using any of a variety of standard techniques, for example, within a chromium release assay or proliferation assay. Alternatively, synthesis of lymphokines (such as IFN-γ) can be measured.

T cells belong to a group of white blood cells known as lymphocytes, and play a central role in cell-mediated immunity. They can be distinguished from other lymphocyte types, such as B cells and natural killer cells by the presence of a special receptor on their cell surface called T cell receptor (TCR). The thymus is the principal organ responsible for the maturation of T cells. Several different subsets of T cells have been discovered, each with a distinct function.

T helper cells assist other white blood cells in immunologic processes, including maturation of B cells into plasma cells and activation of cytotoxic T cells and macrophages, among other functions. These cells are also known as CD4+ T cells because they express the CD4 glycoprotein on their surface. Helper T cells become activated when they are presented with peptide antigens by MHC class II molecules that are expressed on the surface of antigen presenting cells (APCs). Once activated, they divide rapidly and secrete small proteins called cytokines that regulate or assist in the active immune response.

Cytotoxic T cells destroy virally infected cells and tumor cells, and are also implicated in transplant rejection. These cells are also known as CD8+ T cells since they express the CD8 glycoprotein on their surface. These cells recognize their targets by binding to antigen associated with MHC class I, which is present on the surface of nearly every cell of the body.

“Regulatory T cells” or “Tregs” are a subpopulation of T cells that modulate the immune system, maintain tolerance to self-antigens, and prevent autoimmune disease. Tregs are immunosuppressive and generally suppress or downregulate induction and proliferation of effector T cells. Tregs express the biomarkers CD4, FoxP3, and CD25.

As used herein, the term “naïve T cell” refers to mature T cells that, unlike activated or memory T cells, have not encountered their cognate antigen within the periphery. Naïve T cells are commonly characterized by the surface expression of L-selectin (CD62L), the absence of the activation markers CD25, CD44 or CD69 and the absence of the memory CD45RO isoform.

As used herein, the term “memory T cells” refers to a subgroup or subpopulation of T cells that have previously encountered and responded to their cognate antigen. At a second encounter with the antigen, memory T cells can reproduce to mount a faster and stronger immune response than the first time the immune system responded to the antigen. Memory T cells may be either CD4⁺ or CD8⁺ and usually express CD45RO.

All T cells have a T cell receptor (TCR) existing as a complex of several proteins. The TCR of a T cell is able to interact with immunogenic peptides (epitopes) bound to major histocompatibility complex (MHC) molecules and presented on the surface of target cells. Specific binding of the TCR triggers a signal cascade inside the T cell leading to proliferation and differentiation into a maturated effector T cell. In the majority of T cells, the actual T cell receptor is composed of two separate peptide chains, which are produced from the independent T cell receptor alpha and beta (TCRα and TCRβ) genes and are called α- and β-TCR chains. A much less common (2% of total T cells) group of T cells, the γδ T cells (gamma delta T cells) possess a distinct T cell receptor (TCR) on their surface, which is made up of one γ-chain and one δ-chain.

All T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4−CD8−) cells. As they progress through their development they become double-positive thymocytes (CD4+CD8+), and finally mature to single-positive (CD4+CD8− or CD4−CD8+) thymocytes that are then released from the thymus to peripheral tissues.

T cells may generally be prepared in vitro or ex vivo, using standard procedures. For example, T cells may be isolated from bone marrow, peripheral blood or a fraction of bone marrow or peripheral blood of a mammal, such as a patient, using a commercially available cell separation system. Alternatively, T cells may be derived from related or unrelated humans, non-human animals, cell lines or cultures. A sample comprising T cells may, for example, be peripheral blood mononuclear cells (PBMC).

As used herein, the term “NK cell” or “Natural Killer cell” refers to a subset of peripheral blood lymphocytes defined by the expression of CD56 or CD16 and the absence of the T cell receptor. As provided herein, the NK cell can also be differentiated from a stem cell or progenitor cell.

Nucleic Acids

The term “polynucleotide” or “nucleic acid”, as used herein, is intended to include DNA and RNA such as genomic DNA, cDNA, mRNA, recombinantly produced and chemically synthesized molecules. A nucleic acid may be single-stranded or double-stranded. RNA includes in vitro transcribed RNA (IVT RNA) or synthetic RNA. According to the invention, a polynucleotide is preferably isolated.

Nucleic acids may be comprised in a vector. The term “vector” as used herein includes any vectors known to the skilled person including plasmid vectors, cosmid vectors, phage vectors such as lambda phage, viral vectors such as retroviral, adenoviral or baculoviral vectors, or artificial chromosome vectors such as bacterial artificial chromosomes (BAC), yeast artificial chromosomes (YAC), or P1 artificial chromosomes (PAC). Said vectors include expression as well as cloning vectors. Expression vectors comprise plasmids as well as viral vectors and generally contain a desired coding sequence and appropriate DNA sequences necessary for the expression of the operably linked coding sequence in a particular host organism (e.g., bacteria, yeast, plant, insect, or mammal) or in in vitro expression systems. Cloning vectors are generally used to engineer and amplify a certain desired DNA fragment and may lack functional sequences needed for expression of the desired DNA fragments.

In one embodiment of all aspects of the invention, nucleic acid such as nucleic acid encoding a cytokine (e.g., IL2 or IFN), nucleic acid encoding an IL2R, nucleic acid encoding an antigen receptor or nucleic acid encoding a vaccine antigen is expressed in cells of the subject treated to provide the cytokine, IL2R, antigen receptor or vaccine antigen. In one embodiment of all aspects of the invention, the nucleic acid is transiently expressed in cells of the subject. Thus, in one embodiment, the nucleic acid is not integrated into the genome of the cells. In one embodiment of all aspects of the invention, the nucleic acid is RNA, preferably in vitro transcribed RNA.

The nucleic acids described herein may be recombinant and/or isolated molecules.

In the present disclosure, the term “RNA” relates to a nucleic acid molecule which includes ribonucleotide residues. In preferred embodiments, the RNA contains all or a majority of ribonucleotide residues. As used herein, “ribonucleotide” refers to a nucleotide with a hydroxyl group at the 2′-position of a P-D-ribofuranosyl group. RNA encompasses without limitation, double stranded RNA, single stranded RNA, isolated RNA such as partially purified RNA, essentially pure RNA, synthetic RNA, recombinantly produced RNA, as well as modified RNA that differs from naturally occurring RNA by the addition, deletion, substitution and/or alteration of one or more nucleotides. Such alterations may refer to addition of non-nucleotide material to internal RNA nucleotides or to the end(s) of RNA. It is also contemplated herein that nucleotides in RNA may be non-standard nucleotides, such as chemically synthesized nucleotides or deoxynucleotides. For the present disclosure, these altered RNAs are considered analogs of naturally-occurring RNA.

In certain embodiments of the present disclosure, the RNA is messenger RNA (mRNA) that relates to a RNA transcript which encodes a peptide or protein. As established in the art, mRNA generally contains a 5′ untranslated region (5′-UTR), a peptide coding region and a 3′ untranslated region (3′-UTR). In some embodiments, the RNA is produced by in vitro transcription or chemical synthesis. In one embodiment, the mRNA is produced by in vitro transcription using a DNA template where DNA refers to a nucleic acid that contains deoxyribonucleotides.

In one embodiment, RNA is in vitro transcribed RNA (IVT-RNA) and may be obtained by in vitro transcription of an appropriate DNA template. The promoter for controlling transcription can be any promoter for any RNA polymerase. A DNA template for in vitro transcription may be obtained by cloning of a nucleic acid, in particular cDNA, and introducing it into an appropriate vector for in vitro transcription.

The cDNA may be obtained by reverse transcription of RNA.

In one embodiment, the RNA described herein may have modified nucleosides. In some embodiments, the RNA comprises a modified nucleoside in place of at least one (e.g., every) uridine.

The term “uracil,” as used herein, describes one of the nucleobases that can occur in the nucleic acid of RNA. The structure of uracil is:

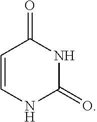

The term “uridine,” as used herein, describes one of the nucleosides that can occur in RNA. The structure of uridine is:

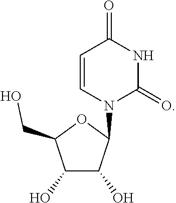 [SMILES: O=c1ccn([C@@H]2O[C@H](CO)[C@@H](O)[C@H]2O)c(=O)[nH]1]

UTP (uridine 5′-triphosphate) has the following structure:

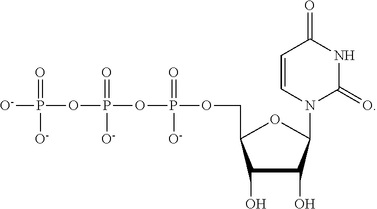

Pseudo-UTP (pseudouridine 5′-triphosphate) has the following structure:

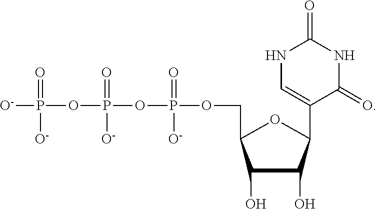

“Pseudouridine” is one example of a modified nucleoside that is an isomer of uridine, where the uracil is attached to the pentose ring via a carbon-carbon bond instead of a nitrogen-carbon glycosidic bond.

Another exemplary modified nucleoside is N1-methyl-pseudouridine (m1ψ), which has the structure:

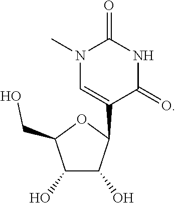 [SMILES: Cn1cc([C@@H]2O[C@H](CO)[C@@H](O)[C@H]2O)c(=O)[nH]c1=O]

N1-methyl-pseudo-UTP has the following structure:

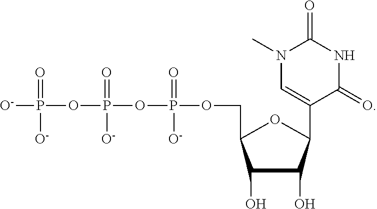

Another exemplary modified nucleoside is 5-methyl-uridine (m5U), which has the structure:

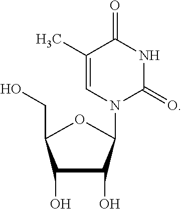

In some embodiments, one or more uridine in the RNA described herein is replaced by a modified nucleoside. In some embodiments, the modified nucleoside is a modified uridine.

In some embodiments, RNA comprises a modified nucleoside in place of at least one uridine. In some embodiments, RNA comprises a modified nucleoside in place of each uridine.

In some embodiments, the modified nucleoside is independently selected from pseudouridine (ψ), N1-methyl-pseudouridine (m1ψ), and 5-methyl-uridine (m5U). In some embodiments, the modified nucleoside comprises pseudouridine (ψ). In some embodiments, the modified nucleoside comprises N1-methyl-pseudouridine (m1ψ). In some embodiments, the modified nucleoside comprises 5-methy-uridine (m5U). In some embodiments, RNA may comprise more than one type of modified nucleoside, and the modified nucleosides are independently selected from pseudouridine (w), N1-methyl-pseudouridine (m1ψ), and 5-methyl-uridine (m5U). In some embodiments, the modified nucleosides comprise pseudouridine (ψ) and N1-methyl-pseudouridine (m1ψ). In some embodiments, the modified nucleosides comprise pseudouridine (ψ) and 5-methyl-uridine (m5U). In some embodiments, the modified nucleosides comprise N1-methyl-pseudouridine (m1ψ) and 5-methyl-uridine (m5U). In some embodiments, the modified nucleosides comprise pseudouridine (ψ), N1-methyl-pseudouridine (m1ψ), and 5-methyl-uridine (m5U).

In some embodiments, the modified nucleoside replacing one or more uridine in the RNA may be any one or more of 3-methyl-uridine (m³U), 5-methoxy-uridine (mo⁵U), 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridine or 5-bromo-uridine), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 1-ethyl-pseudouridine, 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (m⁵U), 1-taurinomethyl-pseudouridine, 5-taurinomethyl-2-thio-uridine (τm5s2U), 1-taurinomethyl-4-thio-pseudouridine), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl-2-thio-uridine (inm⁵s²U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2-O-methyl-uridine (cmnm⁵Um), 3,2′-O-dimethyl-uridine (m³Um), 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, or any other modified uridine known in the art.

In some embodiments, the RNA according to the present disclosure comprises a 5′-cap. In one embodiment, the RNA of the present disclosure does not have uncapped 5′-triphosphates. In one embodiment, the RNA may be modified by a 5′-cap analog. The term “5′-cap” refers to a structure found on the 5′-end of an mRNA molecule and generally consists of a guanosine nucleotide connected to the mRNA via a 5′- to 5′-triphosphate linkage. In one embodiment, this guanosine is methylated at the 7-position. Providing an RNA with a 5′-cap or 5′-cap analog may be achieved by in vitro transcription, in which the 5′-cap is co-transcriptionally expressed into the RNA strand, or may be attached to RNA post-transcriptionally using capping enzymes.

In some embodiments, the building block cap for RNA is m₂^{7,3′-O}Gppp(m₁^{2′-O})ApG (also sometimes referred to as m₂^{7,3′O}G(5′)ppp(5′)m^{2′-O}ApG), which has the following structure:

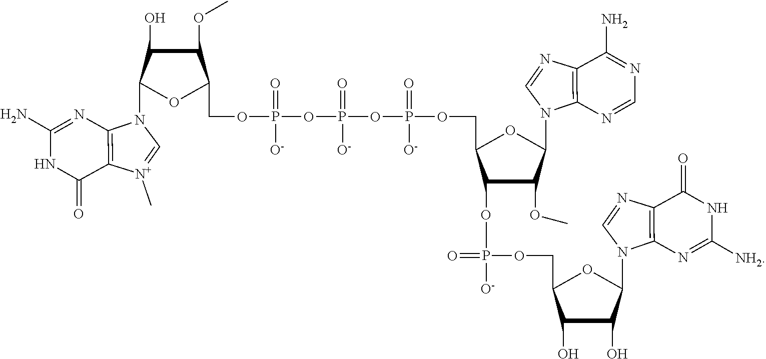

Below is an exemplary Cap1 RNA, which comprises RNA and m₂^{7,3′O}G(5′)ppp(5′)m^{2′-O}ApG:

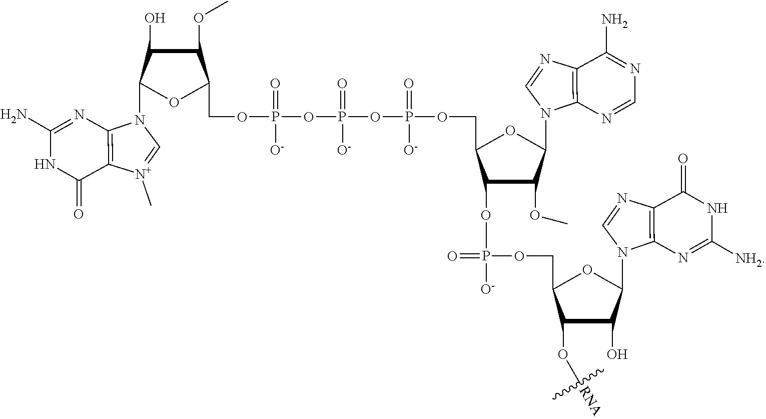

Below is another exemplary Cap1 RNA (no cap analog):

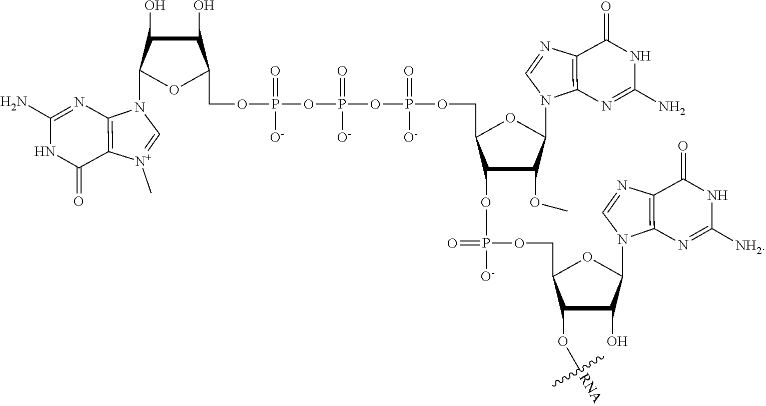

In some embodiments, the RNA is modified with “Cap0” structures using, in one embodiment, the cap analog anti-reverse cap (ARCA Cap (m₂^{7,3′O}G(5′)ppp(5′)G)) with the structure:

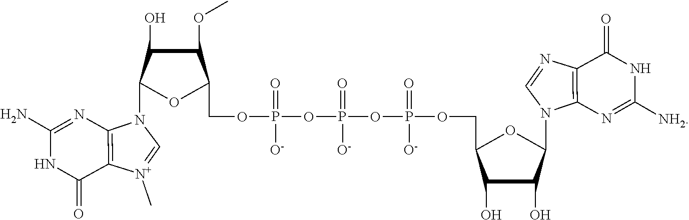

Below is an exemplary Cap0 RNA comprising RNA and m₂^{7,3′O}G(5′)ppp(5′)G:

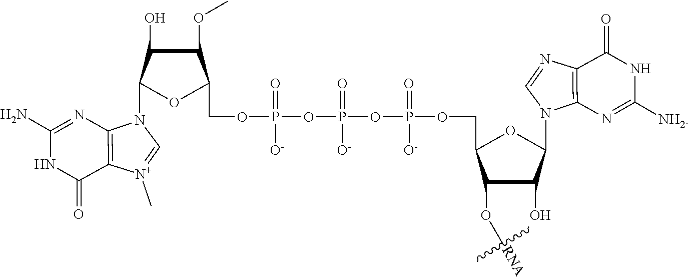

In some embodiments, the “Cap0” structures are generated using the cap analog Beta-S-ARCA (m₂^{7,2′O}G(5′)ppSp(5′)G) with the structure:

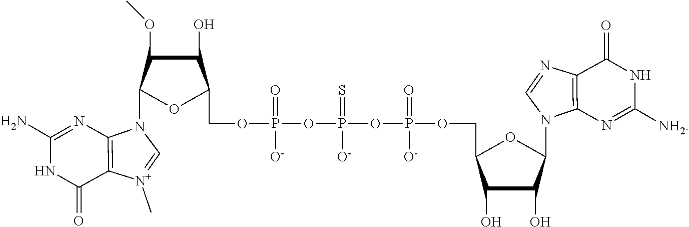

Below is an exemplary Cap0 RNA comprising Beta-S-ARCA (m₂^{7,2′O}G(5′)ppSp(5′)G) and RNA:

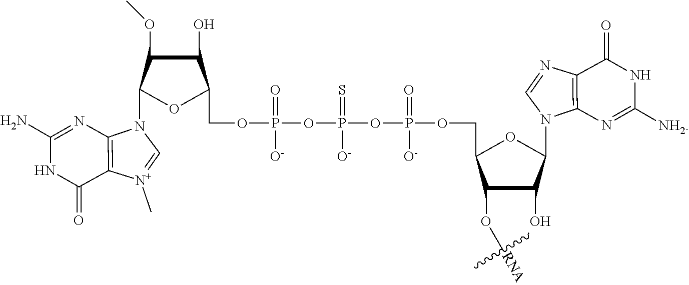

In some embodiments, RNA according to the present disclosure comprises a 5′-UTR and/or a 3′-UTR. The term “untranslated region” or “UTR” relates to a region in a DNA molecule which is transcribed but is not translated into an amino acid sequence, or to the corresponding region in an RNA molecule, such as an mRNA molecule. An untranslated region (UTR) can be present 5′ (upstream) of an open reading frame (5′-UTR) and/or 3′ (downstream) of an open reading frame (3′-UTR). A 5′-UTR, if present, is located at the 5′ end, upstream of the start codon of a protein-encoding region. A 5′-UTR is downstream of the 5′-cap (if present), e.g. directly adjacent to the 5′-cap. A 3′-UTR, if present, is located at the 3′ end, downstream of the termination codon of a protein-encoding region, but the term “3′-UTR” does preferably not include the poly(A) sequence. Thus, the 3′-UTR is upstream of the poly(A) sequence (if present), e.g. directly adjacent to the poly(A) sequence.

In some embodiments, the RNA according to the present disclosure comprises a 3′-poly(A) sequence. As used herein, the term “poly(A) sequence” or “poly-A tail” refers to an uninterrupted or interrupted sequence of adenylate residues which is typically located at the 3′end of an RNA molecule. Poly(A) sequences are known to those of skill in the art and may follow the 3′ UTR in the RNAs described herein. The poly(A) sequence may be of any length. In some embodiments, a poly(A) sequence comprises or consists of at least 20, at least 30, at least 40, at least 80, or at least 100 and up to 500, up to 400, up to 300, up to 200, or up to 150 nucleotides, and, in particular, about 110 nucleotides. In some embodiments, the poly(A) sequence only consists of A nucleotides. In some embodiments, the poly(A) sequence essentially consists of A nucleotides, but is interrupted by a random sequence of the four nucleotides (A, C, G, and U), as disclosed in WO 2016/005324 A1, hereby incorporated by reference. Such random sequence may be 5 to 50, 10 to 30, or 10 to 20 nucleotides in length. A poly(A) cassette present in the coding strand of DNA that essentially consists of dA nucleotides, but is interrupted by a random sequence having an equal distribution of the four nucleotides (dA, dC, dG, dT) and having a length of e.g. 5 to 50 nucleotides shows, on DNA level, constant propagation of plasmid DNA in E. coli and is still associated, on RNA level, with the beneficial properties with respect to supporting RNA stability and translational efficiency. In some embodiments, no nucleotides other than A nucleotides flank a poly(A) sequence at its 3′ end, i.e., the poly(A) sequence is not masked or followed at its 3′end by a nucleotide other than A.

In the context of the present disclosure, the term “transcription” relates to a process, wherein the genetic code in a DNA sequence is transcribed into RNA. Subsequently, the RNA may be translated into peptide or protein.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence.

As used herein, the terms “linked,” “fused”, or “fusion” are used interchangeably. These terms refer to the joining together of two or more elements or components or domains.

Cytokines

Cytokines are a category of small proteins (˜5-20 kDa) that are important in cell signaling. Their release has an effect on the behavior of cells around them. Cytokines are involved in autocrine signaling, paracrine signaling and endocrine signaling as immunomodulating agents. Cytokines include chemokines, interferons, interleukins, lymphokines, and tumour necrosis factors but generally not hormones or growth factors (despite some overlap in the terminology). Cytokines are produced by a broad range of cells, including immune cells like macrophages, B lymphocytes, T lymphocytes and mast cells, as well as endothelial cells, fibroblasts, and various stromal cells. A given cytokine may be produced by more than one type of cell. Cytokines act through receptors, and are especially important in the immune system; cytokines modulate the balance between humoral and cell-based immune responses, and they regulate the maturation, growth, and responsiveness of particular cell populations. Some cytokines enhance or inhibit the action of other cytokines in complex ways.

IL2 and IL2R

Interleukin-2 (IL2) is a cytokine that induces proliferation of antigen-activated T cells and stimulates natural killer (NK) cells. The biological activity of IL2 is mediated through a multi-subunit IL2 receptor complex (IL2R) of three polypeptide subunits that span the cell membrane: p55 (IL2Rα, the alpha subunit, also known as CD25 in humans), p75 (IL2Rβ, the beta subunit, also known as CD122 in humans) and p64 (IL2Rγ, the gamma subunit, also known as CD132 in humans). T cell response to IL2 depends on a variety of factors, including: (1) the concentration of IL2; (2) the number of IL2R molecules on the cell surface; and (3) the number of IL2R occupied by IL2 (i.e., the affinity of the binding interaction between IL2 and IL2R (Smith, “Cell Growth Signal Transduction is Quantal” In Receptor Activation by Antigens, Cytokines, Hormones, and Growth Factors 766:263-271, 1995)). The IL2:IL2R complex is internalized upon ligand binding and the different components undergo differential sorting. When administered as an intravenous (i.v.) bolus, IL2 has a rapid systemic clearance (an initial clearance phase with a half-life of 12.9 minutes followed by a slower clearance phase with a half-life of 85 minutes) (Konrad et al., Cancer Res. 50:2009-2017,1990).

In eukaryotic cells human IL2 is synthesized as a precursor polypeptide of 153 amino acids, from which amino acids are removed to generate mature secreted IL2. Recombinant human IL2 has been produced in E. coli, in insect cells and in mammalian COS cells.

Outcomes of systemic IL2 administration in cancer patients are far from ideal. While 15 to 20 percent of patients respond objectively to high-dose IL2, the great majority do not, and many suffer severe, life-threatening side effects, including nausea, confusion, hypotension, and septic shock. The severe toxicity associated with high-dose IL2 treatment is largely attributable to the activity of natural killer (NK) cells. Attempts to reduce serum concentration by reducing dose and adjusting dosing regimen have been attempted, and while less toxic, such treatments were also less efficacious.

According to the disclosure, IL2 (optionally as a portion of extended-PK IL2) may be naturally occurring IL2 or a fragment or variant thereof. IL2 may be human IL2 and may be derived from any vertebrate, especially any mammal. As used herein, “human IL2” or “wild type human IL2”, whether native or recombinant, has the normally occurring 133 amino acid sequence of native human IL2 (less the signal peptide, consisting of an additional 20 N-terminal amino acids), whose amino acid sequence is described in Fujita, et. al, PNAS USA, 80, 7437-7441 (1983), with or without an additional N-terminal Methionine which is necessarily included when the protein is expressed as an intracellular fraction in E. coli.

In one embodiment, IL2 comprises the amino acid sequence of SEQ ID NO: 1 or 2. In one embodiment, a functional variant of IL2 comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1 or 2. In one embodiment, a functional variant of IL2 binds to the IL2 receptor or a subunit of the IL2 receptor such as the alpha subunit and/or the beta/gamma subunit. In general, for the purposes of this disclosure, the term “IL2” as used herein includes any polypeptide comprising a naturally occurring IL2 moiety or a functional variant thereof, unless contradicted by the circumstances.

According to the disclosure, in certain embodiments, IL2 is attached to a pharmacokinetic modifying group. The resulting molecule, hereafter referred to as “extended-pharmacokinetic (PK) IL2,” has a prolonged circulation half-life relative to free IL2. The prolonged circulation half-life of extended-PK IL2 permits in vivo serum IL2 concentrations to be maintained within a therapeutic range, potentially leading to the enhanced activation of many types of immune cells, including T cells. Because of its favorable pharmacokinetic profile, extended-PK IL2 can be dosed less frequently and for longer periods of time when compared with unmodified IL2.

Accordingly, in certain embodiments described herein, the IL2 moiety is fused to a heterologous polypeptide (i.e., a polypeptide that is not IL2 and preferably is not a variant of IL2) and thus, is extended-PK IL2. In certain embodiments, the IL2 moiety of the extended-PK IL2 is human IL2. In other embodiments, the IL2 moiety of the extended-PK IL2 is a fragment or variant of human IL2. The heterologous polypeptide can increase the circulating half-life of IL2. As discussed in further detail infra, the polypeptide that increases the circulating half-life may be serum albumin, such as human or mouse serum albumin.

According to the disclosure, IL2 receptor (IL2R) may be naturally occurring IL2R or a fragment or variant thereof. IL2R may be human IL2R and may be derived from any vertebrate, especially any mammal. If the IL2 used herein comprises an IL2 variant, the IL2R may be a variant receptor that binds to the IL2 variant.

Interferon

Interferons (IFNs) are a group of signaling proteins made and released by host cells in response to the presence of several pathogens, such as viruses, bacteria, parasites, and also tumor cells. In a typical scenario, a virus-infected cell will release interferons causing nearby cells to heighten their anti-viral defenses.

Based on the type of receptor through which they signal, interferons are typically divided among three classes: type I interferon, type II interferon, and type 11 interferon.

All type I interferons bind to a specific cell surface receptor complex known as the IFN-α/β receptor (IFNAR) that consists of IFNAR1 and IFNAR2 chains.

The type I interferons present in humans are IFNα, IFNβ, IFNε, IFNκ and IFNω. In general, type I interferons are produced when the body recognizes a virus that has invaded it. They are produced by fibroblasts and monocytes. Once released, type I interferons bind to specific receptors on target cells, which leads to expression of proteins that will prevent the virus from producing and replicating its RNA and DNA.

The IFNα proteins are produced mainly by plasmacytoid dendritic cells (pDCs). They are mainly involved in innate immunity against viral infection. The genes responsible for their synthesis come in 13 subtypes that are called IFNA1, IFNA2, IFNA4, IFNA5, IFNA6, IFNA7, IFNA8, IFNA10, IFNA13, IFNA14, IFNA16, IFNA17, IFNA21. These genes are found together in a cluster on chromosome 9.

The IFNβ proteins are produced in large quantities by fibroblasts. They have antiviral activity that is involved mainly in innate immune response. Two types of IFNβ have been described, IFNβ1 and IFNβ3. The natural and recombinant forms of IFNβ1 have antiviral, antibacterial, and anticancer properties.

Type II interferon (IFNγ in humans) is also known as immune interferon and is activated by IL12. Furthermore, type II interferons are released by cytotoxic T cells and T helper cells.

Type III interferons signal through a receptor complex consisting of IL10R2 (also called CRF2-4) and IFNLR1 (also called CRF2-12). Although discovered more recently than type I and type II IFNs, recent information demonstrates the importance of type III IFNs in some types of virus or fungal infections.

In general, type I and II interferons are responsible for regulating and activating the immune response.

According to the disclosure, a type I interferon is preferably IFNα or IFNβ, more preferably IFNα.

According to the disclosure, IFNα may be naturally occurring IFNα or a fragment or variant thereof. IFNα may be human IFNα and may be derived from any vertebrate, especially any mammal. In one embodiment, IFNα comprises the amino acid sequence of SEQ ID NO: 3. In one embodiment, afunctional variant of IFNα comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 3. In one embodiment, afunctional variant of IFNα binds to the IFN-αs receptor. In general, for the purposes of this disclosure, the term “IFNα” as used herein includes any polypeptide comprising a naturally occurring IFNα moiety or a functional variant thereof, unless contradicted by the circumstances.

Extended-PK Group

IL2 polypeptides described herein can be prepared as fusion or chimeric polypeptides that include an IL2 portion and a heterologous polypeptide (i.e., a polypeptide that is not IL2 or a variant thereof). The IL2 may be fused to an extended-PK group, which increases circulation half-life. Non-limiting examples of extended-PK groups are described infra. It should be understood that other PK groups that increase the circulation half-life of cytokines, or variants thereof, are also applicable to the present disclosure. In certain embodiments, the extended-PK group is a serum albumin domain (e.g., mouse serum albumin, human serum albumin).

As used herein, the term “PK” is an acronym for “pharmacokinetic” and encompasses properties of a compound including, by way of example, absorption, distribution, metabolism, and elimination by a subject. As used herein, an “extended-PK group” refers to a protein, peptide, or moiety that increases the circulation half-life of a biologically active molecule when fused to or administered together with the biologically active molecule. Examples of an extended-PK group include serum albumin (e.g., HSA), Immunoglobulin Fc or Fc fragments and variants thereof, transferrin and variants thereof, and human serum albumin (HSA) binders (as disclosed in U.S. Publication Nos. 2005/0287153 and 2007/0003549). Other exemplary extended-PK groups are disclosed in Kontermann, Expert Opin Biol Ther, 2016 July; 16(7):903-15 which is herein incorporated by reference in its entirety. As used herein, an “extended-PK cytokine” refers to a cytokine moiety in combination with an extended-PK group. In one embodiment, the extended-PK cytokine is a fusion protein in which a cytokine moiety is linked or fused to an extended-PK group. As used herein, an “extended-PK IL” refers to an interleukin (IL) moiety (including an IL variant moiety) in combination with an extended-PK group. In one embodiment, the extended-PK IL is a fusion protein in which an IL moiety is linked or fused to an extended-PK group. An exemplary fusion protein is an HSA/IL2 fusion in which an IL2 moiety is fused with HSA.

In certain embodiments, the serum half-life of an extended-PK IL is increased relative to the IL alone (i.e., the IL not fused to an extended-PK group). In certain embodiments, the serum half-life of the extended-PK IL is at least 20, 40, 60, 80, 100, 120, 150, 180, 200, 400, 600, 800, or 1000% longer relative to the serum half-life of the IL alone. In certain embodiments, the serum half-life of the extended-PK IL is at least 1.5-fold, 2-fold, 2.5-fold, 3-fold, 3.5 fold, 4-fold, 4.5-fold, 5-fold, 6-fold, 7-fold, 8-fold, 10-fold, 12-fold, 13-fold, 15-fold, 17-fold, 20-fold, 22-fold, 25-fold, 27-fold, 30-fold, 35-fold, 40-fold, or 50-fold greater than the serum half-life of the IL alone. In certain embodiments, the serum half-life of the extended-PK IL is at least hours, 15 hours, 20 hours, 25 hours, 30 hours, 35 hours, 40 hours, 50 hours, 60 hours, 70 hours, 80 hours, 90 hours, 100 hours, 110 hours, 120 hours, 130 hours, 135 hours, 140 hours, 150 hours, 160 hours, or 200 hours.

As used herein, “half-life” refers to the time taken for the serum or plasma concentration of a compound such as a peptide or protein to reduce by 50%, in vivo, for example due to degradation and/or clearance or sequestration by natural mechanisms. An extended-PK cytokine such as extended-PK interleukin (IL) suitable for use herein is stabilized in vivo and its half-life increased by, e.g., fusion to serum albumin (e.g., HSA or MSA), which resist degradation and/or clearance or sequestration. The half-life can be determined in any manner known per se, such as by pharmacokinetic analysis. Suitable techniques will be clear to the person skilled in the art, and may for example generally involve the steps of suitably administering a suitable dose of the amino acid sequence or compound to a subject; collecting blood samples or other samples from said subject at regular intervals; determining the level or concentration of the amino acid sequence or compound in said blood sample; and calculating, from (a plot of) the data thus obtained, the time until the level or concentration of the amino acid sequence or compound has been reduced by 50% compared to the initial level upon dosing. Further details are provided in, e.g., standard handbooks, such as Kenneth, A. et al., Chemical Stability of Pharmaceuticals: A Handbook for Pharmacists and in Peters et al., Pharmacokinetic Analysis: A Practical Approach (1996). Reference is also made to Gibaldi, M. et al., Pharmacokinetics, 2nd Rev. Edition, Marcel Dekker (1982).

In certain embodiments, the extended-PK group includes serum albumin, or fragments thereof or variants of the serum albumin or fragments thereof (all of which for the purpose of the present disclosure are comprised by the term “albumin”). Polypeptides described herein may be fused to albumin (or a fragment or variant thereof) to form albumin fusion proteins. Such albumin fusion proteins are described in U.S. Publication No. 20070048282.

As used herein, “albumin fusion protein” refers to a protein formed by the fusion of at least one molecule of albumin (or a fragment or variant thereof) to at least one molecule of a protein such as a therapeutic protein, in particular IL2 (or variant thereof). The albumin fusion protein may be generated by translation of a nucleic acid in which a polynucleotide encoding a therapeutic protein is joined in-frame with a polynucleotide encoding an albumin. The therapeutic protein and albumin, once part of the albumin fusion protein, may each be referred to as a “portion”, “region” or “moiety” of the albumin fusion protein (e.g., a “therapeutic protein portion” or an “albumin protein portion”). In a highly preferred embodiment, an albumin fusion protein comprises at least one molecule of a therapeutic protein (including, but not limited to a mature form of the therapeutic protein) and at least one molecule of albumin (including but not limited to a mature form of albumin). In one embodiment, an albumin fusion protein is processed by a host cell such as a cell of the target organ for administered RNA, e.g. a liver cell, and secreted into the circulation. Processing of the nascent albumin fusion protein that occurs in the secretory pathways of the host cell used for expression of the RNA may include, but is not limited to signal peptide cleavage; formation of disulfide bonds; proper folding; addition and processing of carbohydrates (such as for example, N- and O-linked glycosylation); specific proteolytic cleavages; and/or assembly into multimeric proteins. An albumin fusion protein is preferably encoded by RNA in a non-processed form which in particular has a signal peptide at its N-terminus and following secretion by a cell is preferably present in the processed form wherein in particular the signal peptide has been cleaved off. In a most preferred embodiment, the “processed form of an albumin fusion protein” refers to an albumin fusion protein product which has undergone N-terminal signal peptide cleavage, herein also referred to as a “mature albumin fusion protein”.

In preferred embodiments, albumin fusion proteins comprising a therapeutic protein have a higher plasma stability compared to the plasma stability of the same therapeutic protein when not fused to albumin. Plasma stability typically refers to the time period between when the therapeutic protein is administered in vivo and carried into the bloodstream and when the therapeutic protein is degraded and cleared from the bloodstream, into an organ, such as the kidney or liver, that ultimately clears the therapeutic protein from the body. Plasma stability is calculated in terms of the half-life of the therapeutic protein in the bloodstream. The half-life of the therapeutic protein in the bloodstream can be readily determined by common assays known in the art.

As used herein, “albumin” refers collectively to albumin protein or amino acid sequence, or an albumin fragment or variant, having one or more functional activities (e.g., biological activities) of albumin. In particular, “albumin” refers to human albumin or fragments or variants thereof especially the mature form of human albumin, or albumin from other vertebrates or fragments thereof, or variants of these molecules. The albumin may be derived from any vertebrate, especially any mammal, for example human, cow, sheep, or pig. Non-mammalian albumins include, but are not limited to, hen and salmon. The albumin portion of the albumin fusion protein may be from a different animal than the therapeutic protein portion.

In certain embodiments, the albumin is human serum albumin (HSA), or fragments or variants thereof, such as those disclosed in U.S. Pat. No. 5,876,969, WO 2011/124718, WO 2013/075066, and WO 2011/0514789.

The terms, human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, a fragment of albumin sufficient to prolong the therapeutic activity or plasma stability of the therapeutic protein refers to a fragment of albumin sufficient in length or structure to stabilize or prolong the therapeutic activity or plasma stability of the protein so that the plasma stability of the therapeutic protein portion of the albumin fusion protein is prolonged or extended compared to the plasma stability in the non-fusion state.

The albumin portion of the albumin fusion proteins may comprise the full length of the albumin sequence, or may include one or more fragments thereof that are capable of stabilizing or prolonging the therapeutic activity or plasma stability. Such fragments may be of 10 or more amino acids in length or may include about 15, 20, 25, 30, 50, or more contiguous amino acids from the albumin sequence or may include part or all of specific domains of albumin. For instance, one or more fragments of HSA spanning the first two immunoglobulin-like domains may be used. In a preferred embodiment, the HSA fragment is the mature form of HSA.

Generally speaking, an albumin fragment or variant will be at least 100 amino acids long, preferably at least 150 amino acids long.

According to the disclosure, albumin may be naturally occurring albumin or a fragment or variant thereof. Albumin may be human albumin and may be derived from any vertebrate, especially any mammal. In one embodiment, albumin comprises the amino acid sequence of SEQ ID NO: 4 or 5 or an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 4 or 5.

Preferably, the albumin fusion protein comprises albumin as the N-terminal portion, and a therapeutic protein as the C-terminal portion. Alternatively, an albumin fusion protein comprising albumin as the C-terminal portion, and a therapeutic protein as the N-terminal portion may also be used. In other embodiments, the albumin fusion protein has a therapeutic protein fused to both the N-terminus and the C-terminus of albumin. In a preferred embodiment, the therapeutic proteins fused at the N- and C-termini are the same therapeutic proteins. In another preferred embodiment, the therapeutic proteins fused at the N- and C-termini are different therapeutic proteins. In one embodiment, the different therapeutic proteins are both cytokines.

In one embodiment, the therapeutic protein(s) is (are) joined to the albumin through (a) peptide linker(s). A linker peptide between the fused portions may provide greater physical separation between the moieties and thus maximize the accessibility of the therapeutic protein portion, for instance, for binding to its cognate receptor. The linker peptide may consist of amino acids such that it is flexible or more rigid. The linker sequence may be cleavable by a protease or chemically.

As used herein, the term “Fc region” refers to the portion of a native immunoglobulin formed by the respective Fc domains (or Fc moieties) of its two heavy chains. As used herein, the term “Fc domain” refers to a portion or fragment of a single immunoglobulin (Ig) heavy chain wherein the Fc domain does not comprise an Fv domain. In certain embodiments, an Fc domain begins in the hinge region just upstream of the papain cleavage site and ends at the C-terminus of the antibody. Accordingly, a complete Fc domain comprises at least a hinge domain, a CH2 domain, and a CH3 domain. In certain embodiments, an Fc domain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc domain (i.e., a hinge domain, a CH2 domain, and a CH3 domain). In certain embodiments, an Fc domain comprises a hinge domain (or portion thereof) fused to a CH3 domain (or portion thereof). In certain embodiments, an Fc domain comprises a CH2 domain (or portion thereof) fused to a CH3 domain (or portion thereof). In certain embodiments, an Fc domain consists of a CH3 domain or portion thereof. In certain embodiments, an Fc domain consists of a hinge domain (or portion thereof) and a CH3 domain (or portion thereof). In certain embodiments, an Fc domain consists of a CH2 domain (or portion thereof) and a CH3 domain. In certain embodiments, an Fc domain consists of a hinge domain (or portion thereof) and a CH2 domain (or portion thereof). In certain embodiments, an Fc domain lacks at least a portion of a CH2 domain (e.g., all or part of a CH2 domain). An Fc domain herein generally refers to a polypeptide comprising all or part of the Fc domain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc domain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As set forth herein, it will be understood by one of ordinary skill in the art that any Fc domain may be modified such that it varies in amino acid sequence from the native Fc domain of a naturally occurring immunoglobulin molecule. In certain embodiments, the Fc domain has reduced effector function (e.g., FcγR binding).

The Fc domains of a polypeptide described herein may be derived from different immunoglobulin molecules. For example, an Fc domain of a polypeptide may comprise a CH2 and/or CH3 domain derived from an IgG1 molecule and a hinge region derived from an IgG3 molecule. In another example, an Fc domain can comprise a chimeric hinge region derived, in part, from an IgG1 molecule and, in part, from an IgG3 molecule. In another example, an Fc domain can comprise a chimeric hinge derived, in part, from an IgG1 molecule and, in part, from an IgG4 molecule.

In certain embodiments, an extended-PK group includes an Fc domain or fragments thereof or variants of the Fc domain or fragments thereof (all of which for the purpose of the present disclosure are comprised by the term “Fc domain”). The Fc domain does not contain a variable region that binds to antigen. Fc domains suitable for use in the present disclosure may be obtained from a number of different sources.

In certain embodiments, an Fc domain is derived from a human immunoglobulin. In certain embodiments, the Fc domain is from a human IgG1 constant region. It is understood, however, that the Fc domain may be derived from an immunoglobulin of another mammalian species, including for example, a rodent (e.g. a mouse, rat, rabbit, guinea pig) or non-human primate (e.g. chimpanzee, macaque) species.

Moreover, the Fc domain (or a fragment or variant thereof) may be derived from any immunoglobulin class, including IgM, IgG, IgD, IgA, and IgE, and any immunoglobulin isotype, including IgG1, IgG2, IgG3, and IgG4.

A variety of Fc domain gene sequences (e.g., mouse and human constant region gene sequences) are available in the form of publicly accessible deposits. Constant region domains comprising an Fc domain sequence can be selected lacking a particular effector function and/or with a particular modification to reduce immunogenicity. Many sequences of antibodies and antibody-encoding genes have been published and suitable Fc domain sequences (e.g. hinge, CH2, and/or CH3 sequences, or fragments or variants thereof) can be derived from these sequences using art recognized techniques.

In certain embodiments, the extended-PK group is a serum albumin binding protein such as those described in US200510287153, US2007/0003549, US2007/0178082, US2007/0269422, US2010/0113339, WO2009/083804, and WO2009/133208, which are herein incorporated by reference in their entirety. In certain embodiments, the extended-PK group is transferrin, as disclosed in US 7,176,278 and U.S. Pat. No. 8,158,579, which are herein incorporated by reference in their entirety. In certain embodiments, the extended-PK group is a serum immunoglobulin binding protein such as those disclosed in US2007/0178082, US2014/0220017, and US2017/0145062, which are herein incorporated by reference in their entirety. In certain embodiments, the extended-PK group is a fibronectin (Fn)-based scaffold domain protein that binds to serum albumin, such as those disclosed in US2012/0094909, which is herein incorporated by reference in its entirety. Methods of making fibronectin-based scaffold domain proteins are also disclosed in US2012M094909. A non-limiting example of a Fn3-based extended-PK group is Fn3(HSA), i.e., a Fn3 protein that binds to human serum albumin.

In certain aspects, the extended-PK IL, suitable for use according to the disclosure, can employ one or more peptide linkers. As used herein, the term “peptide linker” refers to a peptide or polypeptide sequence which connects two or more domains (e.g., the extended-PK moiety and an IL moiety such as IL2) in a linear amino acid sequence of a polypeptide chain. For example, peptide linkers may be used to connect an IL2 moiety to a HSA domain.

Linkers suitable for fusing the extended-PK group to e.g. IL2 are well known in the art. Exemplary linkers include glycine-serine-polypeptide linkers, glycine-proline-polypeptide linkers, and proline-alanine polypeptide linkers. In certain embodiments, the linker is a glycine-serine-polypeptide linker, i.e., a peptide that consists of glycine and serine residues.

In addition to, or in place of, the heterologous polypeptides described above, an IL2 variant polypeptide described herein can contain sequences encoding a “marker” or “reporter”. Examples of marker or reporter genes include β-lactamase, chloramphenicol acetyltransferase (CAT), adenosine deaminase (ADA), aminoglycoside phosphotransferase, dihydrofolate reductase (DHFR), hygromycin-B-hosphotransferase (HPH), thymidine kinase (TK), β-galactosidase, and xanthine guanine phosphoribosyltransferase (XGPRT).

Antigen Receptors

Immune effector cells described herein may express an antigen receptor such as a T cell receptor (TCR) or chimeric antigen receptor (CAR) binding antigen or a procession product thereof, in particular when present on or presented by a target cell. Cells may naturally express an antigen receptor or be modified (e.g., ex vivo/in vitro or in vivo in a subject to be treated) to express an antigen receptor. Further, immune effector cells may express an IL2R or may be modified (e.g., ex vivo/in vitro or in vivo in a subject to be treated) to express an IL2R. In one embodiment, modification to express an IL2R and modification to express an antigen receptor take place ex vivo/in vitro, either simultaneously or at different time points.

Subsequently, modified cells may be administered to a patient. In one embodiment, modification to express an IL2R takes place ex vivo/in vitro, and, following administration of the cells to a patient, modification to express an antigen receptor takes place in vivo. In one embodiment, modification to express an antigen receptor takes place ex vivo/in vitro, and, following administration of the cells to a patient, modification to express an IL2R takes place in vivo. In one embodiment, modification to express an IL2R and modification to express an antigen receptor takes place in vivo, either simultaneously or at different time points. The cells may be endogenous cells of the patient or may have been administered to a patient.

Chimeric Antigen Receptors

Adoptive cell transfer therapy with CAR-engineered T cells expressing chimeric antigen receptors is a promising anti-cancer therapeutic as CAR-modified T cells can be engineered to target virtually any tumor antigen. For example, patient's T cells may be genetically engineered (genetically modified) to express CARs specifically directed towards antigens on the patient's tumor cells, then infused back into the patient.

According to the invention, the term “CAR” (or “chimeric antigen receptor”) is synonymous with the terms “chimeric T cell receptor” and “artificial T cell receptor” and relates to an artificial receptor comprising a single molecule or a complex of molecules which recognizes, i.e. binds to, a target structure (e.g. an antigen) on a target cell such as a cancer cell (e.g. by binding of an antigen binding domain to an antigen expressed on the surface of the target cell) and may confer specificity onto an immune effector cell such as a T cell expressing said CAR on the cell surface. Preferably, recognition of the target structure by a CAR results in activation of an immune effector cell expressing said CAR. A CAR may comprise one or more protein units said protein units comprising one or more domains as described herein. The term “CAR” does not include T cell receptors.

A CAR comprises a target-specific binding element otherwise referred to as an antigen binding moiety or antigen binding domain that is generally part of the extracellular domain of the CAR. The antigen binding domain recognizes a ligand that acts as a cell surface marker on target cells associated with a particular disease state. Specifically, the CAR of the invention targets the antigen such as tumor antigen on a diseased cell such as tumor cell.

In one embodiment, the binding domain in the CAR binds specifically to the antigen. In one embodiment, the antigen to which the binding domain in the CAR binds is expressed in a cancer cell (tumor antigen). In one embodiment, the antigen is expressed on the surface of a cancer cell. In one embodiment, the binding domain binds to an extracellular domain or to an epitope in an extracellular domain of the antigen. In one embodiment, the binding domain binds to native epitopes of the antigen present on the surface of living cells.

In one embodiment of the invention, an antigen binding domain comprises a variable region of a heavy chain of an immunoglobulin (VH) with a specificity for the antigen and a variable region of a light chain of an immunoglobulin (VL) with a specificity for the antigen. In one embodiment, an immunoglobulin is an antibody. In one embodiment, said heavy chain variable region (VH) and the corresponding light chain variable region (VL) are connected via a peptide linker. Preferably, the antigen binding moiety portion in the CAR is a scFv.

The CAR is designed to comprise a transmembrane domain that is fused to the extracellular domain of the CAR. In one embodiment, the transmembrane domain is not naturally associated with one of the domains in the CAR. In one embodiment, the transmembrane domain is naturally associated with one of the domains in the CAR. In one embodiment, the transmembrane domain is modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain.

In some instances, the CAR of the invention comprises a hinge domain which forms the linkage between the transmembrane domain and the extracellular domain.

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

In one embodiment, the CAR comprises a primary cytoplasmic signaling sequence derived from CD3-zeta. Further, the cytoplasmic domain of the CAR may comprise the CD3-zeta signaling domain combined with a costimulatory signaling region.

The identity of the co-stimulation domain is limited only in that it has the ability to enhance cellular proliferation and survival upon binding of the targeted moiety by the CAR. Suitable co-stimulation domains include CD28, CD137 (4-1BB), a member of the tumor necrosis factor receptor (TNFR) superfamily, CD134 (OX40), a member of the TNFR-superfamily of receptors, and CD278 (ICOS), a CD28-superfamily co-stimulatory molecule expressed on activated T cells. The skilled person will understand that sequence variants of these noted co-stimulation domains can be used without adversely impacting the invention, where the variants have the same or similar activity as the domain on which they are modeled. Such variants will have at least about 80% sequence identity to the amino acid sequence of the domain from which they are derived. In some embodiments of the invention, the CAR constructs comprise two co-stimulation domains. While the particular combinations include all possible variations of the four noted domains, specific examples include CD28+CD137 (4-1BB) and CD28+CD134 (OX40).

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the CAR comprises a signal peptide which directs the nascent protein into the endoplasmic reticulum. In one embodiment, the signal peptide precedes the antigen binding domain. In one embodiment, the signal peptide is derived from an immunoglobulin such as IgG.

The term “antibody” includes an immunoglobulin comprising at least two heavy (H) chains and two light (L) chains inter-connected by disulfide bonds. Each heavy chain is comprised of a heavy chain variable region (abbreviated herein as VH) and a heavy chain constant region. Each light chain is comprised of a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (CIq) of the classical complement system. An antibody binds, preferably specifically binds with an antigen. Antibodies can be intact immunoglobulins derived from natural sources or from recombinant sources and can be immunoreactive portions or fragments of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab′)₂, as well as single chain antibodies and humanized antibodies (Harlow et al., 1999, In: Using Antibodies: A Laboratory Manual, Cold Spring Harbor Laboratory Press, NY; Harlow et al., 1989, in: Antibodies: A Laboratory Manual, Cold Spring Harbor, N.Y.; Houston et al., 1988, Proc. Nat. Acad. Sci. USA 85:5879-5883; Bird et al., 1988, Science 242:423-426).

Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

The term “antibody fragment” refers to a portion of an intact antibody and typically comprises the antigenic determining variable regions of an intact antibody.

Examples of antibody fragments include, but are not limited to, Fab, Fab′, F(ab′)₂, and Fv fragments, linear antibodies, scFv antibodies, and multispecific antibodies formed from antibody fragments.

An “antibody heavy chain”, as used herein, refers to the larger of the two types of polypeptide chains present in antibody molecules in their naturally occurring conformations.

An “antibody light chain”, as used herein, refers to the smaller of the two types of polypeptide chains present in antibody molecules in their naturally occurring conformations, κ and λ light chains refer to the two major antibody light chain isotypes.

According to the disclosure, a CAR which when present on a T cell recognizes an antigen such as on the surface of antigen presenting cells or diseased cells such as cancer cells, such that the T cell is stimulated, and/or expanded or exerts effector functions as described above.

Genetic Modification of Immune Effector Cells

A variety of methods may be used to introduce IL2 receptors and/or antigen receptors such as CAR constructs into cells such as T cells to produce cells genetically modified to express the IL2 receptors and/or antigen receptors. Such methods include non-viral-based DNA transfection, non-viral-based RNA transfection, e.g., mRNA transfection, transposon-based systems, and viral-based systems. Non-viral-based DNA transfection has low risk of insertional mutagenesis. Transposon-based systems can integrate transgenes more efficiently than plasmids that do not contain an integrating element. Viral-based systems include the use of γ-retroviruses and lentiviral vectors. γ-Retroviruses are relatively easy to produce, efficiently and permanently transduce T cells, and have preliminarily proven safe from an integration standpoint in primary human T cells. Lentiviral vectors also efficiently and permanently transduce T cells but are more expensive to manufacture. They are also potentially safer than retrovirus based systems.

In one embodiment of all aspects of the invention, T cells or T cell progenitors are transfected either ex vivo or in vivo with nucleic acid encoding the IL2 receptor and/or nucleic acid encoding the antigen receptor. In one embodiment, a combination of ex vivo and in vivo transfection may be used. In one embodiment of all aspects of the invention, the T cells or T cell progenitors are from the subject to be treated. In one embodiment of all aspects of the invention, the T cells or T cell progenitors are from a subject which is different to the subject to be treated.

CAR T cells may be produced in vivo, and therefore nearly instantaneously, using nanoparticles targeted to T cells. For example, poly(β-amino ester)-based nanoparticles may be coupled to anti-CD3e F(ab) fragments for binding to CD3 on T cells. Upon binding to T cells, these nanoparticles are endocytosed. Their contents, for example plasmid DNA encoding an anti-tumor antigen CAR, may be directed to the T cell nucleus due to the inclusion of peptides containing microtubule-associated sequences (MTAS) and nuclear localization signals (NLSs). The inclusion of transposons flanking the CAR gene expression cassette and a separate plasmid encoding a hyperactive transposase, may allow for the efficient integration of the CAR vector into chromosomes. Such system that allows for the in vivo production of CAR T cells following nanoparticle infusion is described in Smith et al. (2017) Nat. Nanotechnol. 12:813-820.

Another possibility is to use the CRISPR/Cas9 method to deliberately place a CAR coding sequence at a specific locus. For example, existing T cell receptors (TCR) may be knocked out, while knocking in the CAR and placing it under the dynamic regulatory control of the endogenous promoter that would otherwise moderate TCR expression; c.f., e.g., Eyquem et al. (2017) Nature 543:113-117.

In one embodiment of all aspects of the invention, the cells genetically modified to express one or more IL2 receptor polypeptides and/or an antigen receptor are stably or transiently transfected with nucleic acid encoding the IL2 receptor polypeptides and/or nucleic acid encoding the antigen receptor. In one embodiment, the cells are stably transfected with some nucleic acid and transiently transfected with other nucleic acid. Thus, the nucleic acid encoding the IL2 receptor polypeptides and/or the nucleic acid encoding the antigen receptor is integrated or not integrated into the genome of the cells. In one embodiment of all aspects of the invention, the cells genetically modified to express an antigen receptor are inactivated for expression of an endogenous T cell receptor and/or endogenous HLA.

In one embodiment of all aspects of the invention, the cells described herein may be autologous, allogeneic or syngeneic to the subject to be treated. In one embodiment, the present disclosure envisions the removal of cells from a patient and the subsequent re-delivery of the cells to the patient. In one embodiment, the present disclosure does not envision the removal of cells from a patient. In the latter case all steps of genetic modification of cells, if any, may be performed in vivo.

The term “autologous” is used to describe anything that is derived from the same subject. For example, “autologous transplant” refers to a transplant of tissue or organs derived from the same subject. Such procedures are advantageous because they overcome the immunological barrier which otherwise results in rejection.

The term “allogeneic” is used to describe anything that is derived from different individuals of the same species. Two or more individuals are said to be allogeneic to one another when the genes at one or more loci are not identical.

The term “syngeneic” is used to describe anything that is derived from individuals or tissues having identical genotypes, i.e., identical twins or animals of the same inbred strain, or their tissues.

The term “heterologous” is used to describe something consisting of multiple different elements. As an example, the transfer of one individual's bone marrow into a different individual constitutes a heterologous transplant. A heterologous gene is a gene derived from a source other than the subject.

Antigen

In one embodiment, the methods described herein further comprise the step of contacting the immune effector cells, in particular immune effector cells expressing an antigen receptor, e.g., immune effector cells which are genetically manipulated to express an antigen receptor, either ex vivo or in the subject being treated, with a cognate antigen molecule, wherein the antigen molecule or a procession product thereof, e.g., a fragment thereof, binds to the antigen receptor such as TCR or CAR carried by the immune effector cells. Such cognate antigen molecule is also designated herein as “vaccine antigen” or “peptide or protein comprising an epitope for inducing an immune response against an antigen”. In one embodiment, the cognate antigen molecule is selected from the group consisting of the antigen expressed by a target cell to which the immune effector cells are targeted or a fragment thereof, or a variant of the antigen or the fragment. In one embodiment, the immune effector cells are contacted with the cognate antigen molecule under conditions such that expansion and/or activation of the immune effector cells occurs. In one embodiment, the step of contacting the immune effector cells with the cognate antigen molecule takes place in vivo or ex vivo.

In one embodiment, the methods described herein comprise the step of administering the cognate antigen molecule or a nucleic acid coding therefor to the subject. In one embodiment, the nucleic acid encoding the cognate antigen molecule is expressed in cells of the subject to provide the cognate antigen molecule. In one embodiment, expression of the cognate antigen molecule is at the cell surface. In one embodiment, the nucleic acid encoding the cognate antigen molecule is transiently expressed in cells of the subject. In one embodiment, the nucleic encoding the cognate antigen molecule is RNA. In one embodiment, the cognate antigen molecule or the nucleic acid coding therefor is administered systemically. In one embodiment, after systemic administration of the nucleic acid encoding the cognate antigen molecule, expression of the nucleic acid encoding the cognate antigen molecule in spleen occurs. In one embodiment, after systemic administration of the nucleic acid encoding the cognate antigen molecule, expression of the nucleic acid encoding the cognate antigen molecule in antigen presenting cells, preferably professional antigen presenting cells occurs. In one embodiment, the antigen presenting cells are selected from the group consisting of dendritic cells, macrophages and B cells. In one embodiment, after systemic administration of the nucleic acid encoding the cognate antigen molecule, no or essentially no expression of the nucleic acid encoding the cognate antigen molecule in lung and/or liver occurs. In one embodiment, after systemic administration of the nucleic acid encoding the cognate antigen molecule, expression of the nucleic acid encoding the cognate antigen molecule in spleen is at least 5-fold the amount of expression in lung.

A peptide and protein antigen which is provided to a subject according to the invention (either by administering the peptide and protein antigen or a nucleic acid, in particular RNA, encoding the peptide and protein antigen), i.e., a vaccine antigen, preferably results in stimulation, priming and/or expansion of immune effector cells in the subject being administered the peptide or protein antigen or nucleic acid. Said stimulated, primed and/or expanded immune effector cells are preferably directed against a target antigen, in particular a target antigen expressed by diseased cells, tissues and/or organs, i.e., a disease-associated antigen. Thus, a vaccine antigen may comprise the disease-associated antigen, or a fragment or variant thereof. In one embodiment, such fragment or variant is immunologically equivalent to the disease-associated antigen. In the context of the present disclosure, the term “fragment of an antigen” or “variant of an antigen” means an agent which results in stimulation, priming and/or expansion of immune effector cells which stimulated, primed and/or expanded immune effector cells target the antigen, i.e. a disease-associated antigen, in particular when presented by diseased cells, tissues and/or organs. Thus, the vaccine antigen may correspond to or may comprise the disease-associated antigen, may correspond to or may comprise a fragment of the disease-associated antigen or may correspond to or may comprise an antigen which is homologous to the disease-associated antigen or a fragment thereof. If the vaccine antigen comprises a fragment of the disease-associated antigen or an amino acid sequence which is homologous to a fragment of the disease-associated antigen said fragment or amino acid sequence may comprise an epitope of the disease-associated antigen to which the antigen receptor of the immune effector cells is targeted or a sequence which is homologous to an epitope of the disease-associated antigen. Thus, according to the disclosure, a vaccine antigen may comprise an immunogenic fragment of a disease-associated antigen or an amino acid sequence being homologous to an immunogenic fragment of a disease-associated antigen. An “immunogenic fragment of an antigen” according to the disclosure preferably relates to a fragment of an antigen which is capable of stimulating, priming and/or expanding immune effector cells carrying an antigen receptor binding to the antigen or cells expressing the antigen. It is preferred that the vaccine antigen (similar to the disease-associated antigen) provides the relevant epitope for binding by the antigen binding domain present in the immune effector cells. In one embodiment, the vaccine antigen (similar to the disease-associated antigen) is expressed on the surface of a cell such as an antigen-presenting cell so as to provide the relevant epitope for binding by immune effector cells. The vaccine antigen may be a recombinant antigen.

In one embodiment of all aspects of the invention, the nucleic acid encoding the vaccine antigen is expressed in cells of a subject to provide the antigen or a procession product thereof for binding by the antigen receptor expressed by immune effector cells, said binding resulting in stimulation, priming and/or expansion of the immune effector cells.

The term “immunologically equivalent” means that the immunologically equivalent molecule such as the immunologically equivalent amino acid sequence exhibits the same or essentially the same immunological properties and/or exerts the same or essentially the same immunological effects, e.g., with respect to the type of the immunological effect. In the context of the present disclosure, the term “immunologically equivalent” is preferably used with respect to the immunological effects or properties of antigens or antigen variants used for immunization. For example, an amino acid sequence is immunologically equivalent to a reference amino acid sequence if said amino acid sequence when exposed to the immune system of a subject such as T cells binding to the reference amino acid sequence or cells expressing the reference amino acid sequence induces an immune reaction having a specificity of reacting with the reference amino acid sequence. Thus, a molecule which is immunologically equivalent to an antigen exhibits the same or essentially the same properties and/or exerts the same or essentially the same effects regarding the stimulation, priming and/or expansion of T cells as the antigen to which the T cells are targeted.

“Activation” or “stimulation”, as used herein, refers to the state of an immune effector cell such as T cell that has been sufficiently stimulated to induce detectable cellular proliferation. Activation can also be associated with initiation of signaling pathways, induced cytokine production, and detectable effector functions. The term “activated immune effector cells” refers to, among other things, immune effector cells that are undergoing cell division.

The term “priming” refers to a process wherein an immune effector cell such as a T cell has its first contact with its specific antigen and causes differentiation into effector cells such as effector T cells.

The term “clonal expansion” or “expansion” refers to a process wherein a specific entity is multiplied. In the context of the present disclosure, the term is preferably used in the context of an immunological response in which lymphocytes are stimulated by an antigen, proliferate, and the specific lymphocyte recognizing said antigen is amplified. Preferably, clonal expansion leads to differentiation of the lymphocytes.

The term “antigen” relates to an agent comprising an epitope against which an immune response can be generated. The term “antigen” includes, in particular, proteins and peptides. In one embodiment, an antigen is presented or present on the surface of cells of the immune system such as antigen presenting cells like dendritic cells or macrophages. An antigen or a procession product thereof such as a T cell epitope is in one embodiment bound by an antigen receptor. Accordingly, an antigen or a procession product thereof may react specifically with immune effector cells such as T-lymphocytes (T cells). In one embodiment, an antigen is a disease-associated antigen, such as a tumor antigen, a viral antigen, or a bacterial antigen and an epitope is derived from such antigen.

The term “disease-associated antigen” is used in its broadest sense to refer to any antigen associated with a disease. A disease-associated antigen is a molecule which contains epitopes that will stimulate a hosts immune system to make a cellular antigen-specific immune response and/or a humoral antibody response against the disease. The disease-associated antigen or an epitope thereof may therefore be used for therapeutic purposes. Disease-associated antigens may be associated with infection by microbes, typically microbial antigens, or associated with cancer, typically tumors.

The term “tumor antigen” refers to a constituent of cancer cells which may be derived from the cytoplasm, the cell surface and the cell nucleus. In particular, it refers to those antigens which are produced intracellularly or as surface antigens on tumor cells. A tumor antigen is typically expressed preferentially by cancer cells (e.g., it is expressed at higher levels in cancer cells than in non-cancer cells) and in some instances it is expressed solely by cancer cells. Examples of tumor antigens include, without limitation, p53, ART-4, BAGE, beta-catenin/m, Bcr-abL CAMEL, CAP-1, CASP-8, CDCl₂7/m, CDK4/m, CEA, the cell surface proteins of the claudin family, such as CLAUDIN-6, CLAUDIN-18.2 and CLAUDIN-12, c-MYC, CT, Cyp-B, DAM, ELF2M, ETV6-AML1, G250, GAGE, GnT-V, Gap 100, HAGE, HER-2/neu, HPV-E7, HPV-E6, HAST-2, hTERT (or hTRT), LAGE, LDLR/FUT, MAGE-A, preferably MAGE-A1, MAGE-A2, MAGE-A3, MAGE-A4, MAGE-A5, MAGE-A6, MAGE-A7, MAGE-A8, MAGE-A9, MAGE-A 10, MAGE-A 11, or MAGE-A12, MAGE-B, MAGE-C, MART-1/Melan-A, MC1R, Myosin/m, MUC1, MUM-1, MUM-2, MUM-3, NA88-A, NF1, NY-ESO-1, NY-BR-1, p190 minor BCR-abL, Pml/RARa, PRAME, proteinase 3, PSA, PSM, RAGE, RU1 or RU2, SAGE, SART-1 or SART-3, SCGB3A2, SCP1, SCP2, SCP3, SSX, SURVIVIN, TEL/AML1, TPI/m, TRP-1, TRP-2, TRP-2/INT2, TPTE, WT, and WT-1.

The term “viral antigen” refers to any viral component having antigenic properties, i.e. being able to provoke an immune response in an individual. The viral antigen may be a viral ribonucleoprotein or an envelope protein.

The term “bacterial antigen” refers to any bacterial component having antigenic properties, i.e. being able to provoke an immune response in an individual. The bacterial antigen may be derived from the cell wall or cytoplasm membrane of the bacterium.

The term “expressed on the cell surface” or “associated with the cell surface” means that a molecule such as a receptor or antigen is associated with and located at the plasma membrane of a cell, wherein at least a part of the molecule faces the extracellular space of said cell and is accessible from the outside of said cell, e.g., by antibodies located outside the cell. In this context, a part is preferably at least 4, preferably at least 8, preferably at least 12, more preferably at least 20 amino acids. The association may be direct or indirect. For example, the association may be by one or more transmembrane domains, one or more lipid anchors, or by the interaction with any other protein, lipid, saccharide, or other structure that can be found on the outer leaflet of the plasma membrane of a cell. For example, a molecule associated with the surface of a cell may be a transmembrane protein having an extracellular portion or may be a protein associated with the surface of a cell by interacting with another protein that is a transmembrane protein.

“Cell surface” or “surface of a cell” is used in accordance with its normal meaning in the art, and thus includes the outside of the cell which is accessible to binding by proteins and other molecules.

The term “extracellular portion” or “exodomain” in the context of the present invention refers to a part of a molecule such as a protein that is facing the extracellular space of a cell and preferably is accessible from the outside of said cell, e.g., by binding molecules such as antibodies located outside the cell. Preferably, the term refers to one or more extracellular loops or domains or a fragment thereof.

The term “epitope” refers to a part or fragment of a molecule such as an antigen that is recognized by the immune system. For example, the epitope may be recognized by T cells, B cells or antibodies. An epitope of an antigen may include a continuous or discontinuous portion of the antigen and may be between about and about 100, such as between about 5 and about 50, more preferably between about 8 and about 30, most preferably between about 10 and about 25 amino acids in length, for example, the epitope may be preferably 9, 10, 11, 12, 13, 14, 15, 16, 17, 18, 19, 20, 21, 22, 23, 24, or 25 amino acids in length. In one embodiment, an epitope is between about 10 and about 25 amino acids in length. The term “epitope” includes T cell epitopes.

The term “T cell epitope” refers to a part or fragment of a protein that is recognized by a T cell when presented in the context of MHC molecules. The term “major histocompatibility complex” and the abbreviation “MHC” includes MHC class I and MHC class II molecules and relates to a complex of genes which is present in all vertebrates. MHC proteins or molecules are important for signaling between lymphocytes and antigen presenting cells or diseased cells in immune reactions, wherein the MHC proteins or molecules bind peptide epitopes and present them for recognition by T cell receptors on T cells. The proteins encoded by the MHC are expressed on the surface of cells, and display both self-antigens (peptide fragments from the cell itself) and non-self-antigens (e.g., fragments of invading microorganisms) to a T cell. In the case of class I MHC/peptide complexes, the binding peptides are typically about 8 to about 10 amino acids long although longer or shorter peptides may be effective. In the case of class II MHC/peptide complexes, the binding peptides are typically about 10 to about 25 amino acids long and are in particular about 13 to about 18 amino acids long, whereas longer and shorter peptides may be effective.

In one embodiment, the target antigen is a tumor antigen and the vaccine antigen or a fragment thereof (e.g., an epitope) is derived from the tumor antigen. The tumor antigen may be a “standard” antigen, which is generally known to be expressed in various cancers. The tumor antigen may also be a “neo-antigen”, which is specific to an individual's tumor and has not been previously recognized by the immune system. A neo-antigen or neo-epitope may result from one or more cancer-specific mutations in the genome of cancer cells resulting in amino acid changes. If the tumor antigen is a neo-antigen, the vaccine antigen preferably comprises an epitope or a fragment of said neo-antigen comprising one or more amino acid changes.

Cancer mutations vary with each individual. Thus, cancer mutations that encode novel epitopes (neo-epitopes) represent attractive targets in the development of vaccine compositions and immunotherapies. The efficacy of tumor immunotherapy relies on the selection of cancer-specific antigens and epitopes capable of inducing a potent immune response within a host. RNA can be used to deliver patient-specific tumor epitopes to a patient. Dendritic cells (DCs) residing in the spleen represent antigen-presenting cells of particular interest for RNA expression of immunogenic epitopes or antigens such as tumor epitopes.

The use of multiple epitopes has been shown to promote therapeutic efficacy in tumor vaccine compositions. Rapid sequencing of the tumor mutanome may provide multiple epitopes for individualized vaccines which can be encoded by RNA described herein, e.g., as a single polypeptide wherein the epitopes are optionally separated by linkers. In certain embodiments of the present disclosure, the RNA encodes at least one epitope, at least two epitopes, at least three epitopes, at least four epitopes, at least five epitopes, at least six epitopes, at least seven epitopes, at least eight epitopes, at least nine epitopes, or at least ten epitopes. Exemplary embodiments include RNA that encodes at least five epitopes (termed a “pentatope”) and RNA that encodes at least ten epitopes (termed a “decatope”).

According to the various aspects of the invention, the aim is preferably to provide an immune response against cancer cells expressing a tumor antigen and to treat a cancer disease involving cells expressing a tumor antigen. In one embodiment, the invention involves the administration of antigen receptor-engineered immune effector cells such as T cells targeted against cancer cells expressing a tumor antigen.

The peptide and protein antigen can be 2-100 amino acids, including for example, 5 amino acids, 10 amino acids, 15 amino acids, 20 amino acids, 25 amino acids, 30 amino acids, 35 amino acids, 40 amino acids, 45 amino acids, or 50 amino acids in length. In some embodiments, a peptide can be greater than 50 amino acids. In some embodiments, the peptide can be greater than 100 amino acids.

According to the invention, the vaccine antigen should be recognizable by an immune effector cell. Preferably, the antigen if recognized by an immune effector cell is able to induce in the presence of appropriate co-stimulatory signals, stimulation, priming and/or expansion of the immune effector cell carrying an antigen receptor recognizing the antigen. In the context of the embodiments of the present invention, the antigen is preferably present on the surface of a cell, preferably an antigen presenting cell. Recognition of the antigen on the surface of a diseased cell may result in an immune reaction against the antigen (or cell expressing the antigen).

In one embodiment of all aspects of the invention, an antigen is expressed in a diseased cell such as a cancer cell. In one embodiment, an antigen is expressed on the surface of a diseased cell such as a cancer cell. In one embodiment, an antigen receptor is a CAR which binds to an extracellular domain or to an epitope in an extracellular domain of an antigen. In one embodiment, a CAR binds to native epitopes of an antigen present on the surface of living cells. In one embodiment, binding of a CAR when expressed by T cells and/or present on T cells to an antigen present on cells such as antigen presenting cells results in stimulation, priming and/or expansion of said T cells. In one embodiment, binding of a CAR when expressed by T cells and/or present on T cells to an antigen present on diseased cells such as cancer cells results in cytolysis and/or apoptosis of the diseased cells, wherein said T cells preferably release cytotoxic factors, e.g. perforins and granzymes.

Immune Checkpoint Inhibitors

In certain embodiments, immune checkpoint inhibitors are used in combination with other therapeutic agents described herein.

As used herein, “immune checkpoint” refers to co-stimulatory and inhibitory signals that regulate the amplitude and quality of T cell receptor recognition of an antigen. In certain embodiments, the immune checkpoint is an inhibitory signal. In certain embodiments, the inhibitory signal is the interaction between PD-1 and PD-L1. In certain embodiments, the inhibitory signal is the interaction between CTLA-4 and CD80 or CD86 to displace CD28 binding. In certain embodiments the inhibitory signal is the interaction between LAG3 and MHC class II molecules. In certain embodiments, the inhibitory signal is the interaction between TIM3 and galectin 9.

As used herein, “immune checkpoint inhibitor” refers to a molecule that totally or partially reduces, inhibits, interferes with or modulates one or more checkpoint proteins. In certain embodiments, the immune checkpoint inhibitor prevents inhibitory signals associated with the immune checkpoint. In certain embodiments, the immune checkpoint inhibitor is an antibody, or fragment thereof that disrupts inhibitory signaling associated with the immune checkpoint. In certain embodiments, the immune checkpoint inhibitor is a small molecule that disrupts inhibitory signaling. In certain embodiments, the immune checkpoint inhibitor is an antibody, fragment thereof, or antibody mimic, that prevents the interaction between checkpoint blocker proteins, e.g., an antibody, or fragment thereof, that prevents the interaction between PD-1 and PD-L1. In certain embodiments, the immune checkpoint inhibitor is an antibody, or fragment thereof, that prevents the interaction between CTLA-4 and CD80 or CD86. In certain embodiments, the immune checkpoint inhibitor is an antibody, or fragment thereof, that prevents the interaction between LAG3 and its ligands, or TIM-3 and its ligands. The checkpoint inhibitor may also be in the form of the soluble form of the molecules (or variants thereof) themselves, e.g., a soluble PD-L1 or PD-L1 fusion.

The “Programmed Death-1 (PD-1)” receptor refers to an immuno-inhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1.

“Programmed Death Ligand-1 (PD-1)” is one of two cell surface glycoprotein ligands for PD-1 (the other being PD-L2) that downregulates T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1.

“Cytotoxic T Lymphocyte Associated Antigen-4 (CTLA-4)” is a T cell surface molecule and is a member of the immunoglobulin superfamily. This protein downregulates the immune system by binding to CD80 and CD86. The term “CTLA-4” as used herein includes human CTLA-4 (hCTLA-4), variants, isoforms, and species homologs of hCTLA-4, and analogs having at least one common epitope with hCTLA-4.

“Lymphocyte Activation Gene-3 (LAG3)” is an inhibitory receptor associated with inhibition of lymphocyte activity by binding to MHC class II molecules. This receptor enhances the function of Treg cells and inhibits CD8+ effector T cell function. The term “LAG3” as used herein includes human LAG3 (hLAG3), variants, isoforms, and species homologs of hLAG3, and analogs having at least one common epitope.

“T Cell Membrane Protein-3 (TIM3)” is an inhibitory receptor involved in the inhibition of lymphocyte activity by inhibition of TH1 cell responses. Its ligand is galectin 9, which is upregulated in various types of cancers. The term “TIM3” as used herein includes human TIM3 (hTIM3), variants, isoforms, and species homologs of hTIM3, and analogs having at least one common epitope.

The “B7 family” refers to inhibitory ligands with undefined receptors. The B7 family encompasses B7-H3 and B7-H4, both upregulated on tumor cells and tumor infiltrating cells.

In certain embodiments, the immune checkpoint inhibitor suitable for use in the methods disclosed herein, is an antagonist of inhibitory signals, e.g., an antibody which targets, for example, PD-1, PD-L1, CTLA-4, LAG3, B7-H3, B7-H4, or TIM3. These ligands and receptors are reviewed in Pardoll, D., Nature. 12: 252-264, 2012.

In certain embodiments, the immune checkpoint inhibitor is an antibody or an antigen-binding portion thereof, that disrupts or inhibits signaling from an inhibitory immunoregulator. In certain embodiments, the immune checkpoint inhibitor is a small molecule that disrupts or inhibits signaling from an inhibitory immunoregulator.

In certain embodiments, the inhibitory immunoregulator is a component of the PD-1/PD-L1 signaling pathway. Accordingly, certain embodiments of the disclosure provide for administering to a subject an antibody or an antigen-binding portion thereof that disrupts the interaction between the PD-1 receptor and its ligand, PD-L1. Antibodies which bind to PD-1 and disrupt the interaction between the PD-1 and its ligand, PD-L1, are known in the art. In certain embodiments, the antibody or antigen-binding portion thereof binds specifically to PD-1. In certain embodiments, the antibody or antigen-binding portion thereof binds specifically to PD-L1 and inhibits its interaction with PD-1, thereby increasing immune activity.

In certain embodiments, the inhibitory immunoregulator is a component of the CTLA4 signaling pathway. Accordingly, certain embodiments of the disclosure provide for administering to a subject an antibody or an antigen-binding portion thereof that targets CTLA4 and disrupts its interaction with CD80 and CD86.

In certain embodiments, the inhibitory immunoregulator is a component of the LAG3 (lymphocyte activation gene 3) signaling pathway. Accordingly, certain embodiments of the disclosure provide for administering to a subject an antibody or an antigen-binding portion thereof that targets LAG3 and disrupts its interaction with MHC class II molecules.

In certain embodiments, the inhibitory immunoregulator is a component of the B7 family signaling pathway. In certain embodiments, the B7 family members are B7-H3 and B7-H4. Accordingly, certain embodiments of the disclosure provide for administering to a subject an antibody or an antigen-binding portion thereof that targets B7-H3 or H4. The B7 family does not have any defined receptors but these ligands are upregulated on tumor cells or tumor-infiltrating cells. Preclinical mouse models have shown that blockade of these ligands can enhance anti-tumor immunity.

In certain embodiments, the inhibitory immunoregulator is a component of the TIM3 (T cell membrane protein 3) signaling pathway. Accordingly, certain embodiments of the disclosure provide for administering to a subject an antibody or an antigen-binding portion thereof that targets TIM3 and disrupts its interaction with galectin 9.

It will be understood by one of ordinary skill in the art that other immune checkpoint targets can also be targeted by antagonists or antibodies, provided that the targeting results in the stimulation of an immune response such as an anti-tumor immune response as reflected in, e.g., an increase in T cell proliferation, enhanced T cell activation, and/or increased cytokine production (e.g., IFN-γ, IL2).

RNA Targeting

It is particularly preferred according to the invention that the peptides, proteins or polypeptides described herein, in particular the IL2 polypeptides, IFN polypeptides and/or vaccine antigens, are administered in the form of RNA encoding the peptides, proteins or polypeptides described herein. In one embodiment, different peptides, proteins or polypeptides described herein are encoded by different RNA molecules.

In one embodiment, the RNA is formulated in a delivery vehicle. In one embodiment, the delivery vehicle comprises particles. In one embodiment, the delivery vehicle comprises at least one lipid. In one embodiment, the at least one lipid comprises at least one cationic lipid. In one embodiment, the lipid forms a complex with and/or encapsulates the RNA. In one embodiment, the lipid is comprised in a vesicle encapsulating the RNA. In one embodiment, the RNA is formulated in liposomes.

According to the disclosure, after administration of the RNA described herein, at least a portion of the RNA is delivered to a target cell. In one embodiment, at least a portion of the RNA is delivered to the cytosol of the target cell. In one embodiment, the RNA is translated by the target cell to produce the encoded peptide or protein.

Some aspects of the disclosure involve the targeted delivery of the RNA disclosed herein (e.g., RNA encoding IL2 polypeptides, RNA encoding IFN polypeptides and/or RNA encoding vaccine antigen).

In one embodiment, the disclosure involves targeting the lymphatic system, in particular secondary lymphoid organs, more specifically spleen. Targeting the lymphatic system, in particular secondary lymphoid organs, more specifically spleen is in particular preferred if the RNA administered is RNA encoding vaccine antigen.

In one embodiment, the target cell is a spleen cell. In one embodiment, the target cell is an antigen presenting cell such as a professional antigen presenting cell in the spleen. In one embodiment, the target cell is a dendritic cell in the spleen.

The “lymphatic system” is part of the circulatory system and an important part of the immune system, comprising a network of lymphatic vessels that carry lymph. The lymphatic system consists of lymphatic organs, a conducting network of lymphatic vessels, and the circulating lymph. The primary or central lymphoid organs generate lymphocytes from immature progenitor cells. The thymus and the bone marrow constitute the primary lymphoid organs. Secondary or peripheral lymphoid organs, which include lymph nodes and the spleen, maintain mature naïve lymphocytes and initiate an adaptive immune response.

RNA may be delivered to spleen by so-called lipoplex formulations, in which the RNA is bound to liposomes comprising a cationic lipid and optionally an additional or helper lipid to form injectable nanoparticle formulations. The liposomes may be obtained by injecting a solution of the lipids in ethanol into water or a suitable aqueous phase. RNA lipoplex particles may be prepared by mixing the liposomes with RNA. Spleen targeting RNA lipoplex particles are described in WO 2013/143683, herein incorporated by reference. It has been found that RNA lipoplex particles having a net negative charge may be used to preferentially target spleen tissue or spleen cells such as antigen-presenting cells, in particular dendritic cells. Accordingly, following administration of the RNA lipoplex particles, RNA accumulation and/or RNA expression in the spleen occurs. Thus, RNA lipoplex particles of the disclosure may be used for expressing RNA in the spleen. In an embodiment, after administration of the RNA lipoplex particles, no or essentially no RNA accumulation and/or RNA expression in the lung and/or liver occurs. In one embodiment, after administration of the RNA lipoplex particles, RNA accumulation and/or RNA expression in antigen presenting cells, such as professional antigen presenting cells in the spleen occurs. Thus, RNA lipoplex particles of the disclosure may be used for expressing RNA in such antigen presenting cells. In one embodiment, the antigen presenting cells are dendritic cells and/or macrophages.

In the context of the present disclosure, the term “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA, and additional lipids, such as DOPE. In one embodiment, a RNA lipoplex particle is a nanoparticle.

As used herein, a “cationic lipid” refers to a lipid having a net positive charge. Cationic lipids bind negatively charged RNA by electrostatic interaction to the lipid matrix. Generally, cationic lipids possess alipophilic moiety, such as a sterol, an acyl or diacyl chain, and the head group of the lipid typically carries the positive charge. Examples of cationic lipids include, but are not limited to 1,2-di-o-octadecenyl-3-trimethylammonium propane (DOTMA), dimethyldioctadecylammonium (DDAB); 1,2-dioleoyl-3-trimethylammonium propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 2,3-di(tetradecoxy)propyl-(2-hydroxyethyl)-dimethylazanium (DMRIE), 1,2-dimyristoyl-sn-glycero-3-ethylphosphocholine (DMEPC), 1,2-dimyristoyl-3-trimethylammonium propane (DMTAP), 1,2-dioleyloxypropyl-3-dimethyl-hydroxyethyl ammonium bromide (DORIE), and 2,3-dioleoyloxy-N-[2(spermine carboxamide)ethyl]-N,N-dimethyl-I-propanamium trifluoroacetate (DOSPA). Preferred are DOTMA, DOTAP, DODAC, and DOSPA. In specific embodiments, the cationic lipid is DOTMA and/or DOTAP.

An additionallipid may be incorporated to adjust the overall positive to negative charge ratio and physical stability of the RNA lipoplex particles. In certain embodiments, the additional lipid is a neutral lipid. As used herein, a “neutral lipid” refers to a lipid having a net charge of zero. Examples of neutral lipids include, but are not limited to, 1,2-di-(9Z-octadecenoyl)-sn-glycero-3-phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, cholesterol, and cerebroside. In specific embodiments, the additional lipid is DOPE, cholesterol and/or DOPC.

In certain embodiments, the RNA lipoplex particles include both a cationic lipid and an additional lipid. In an exemplary embodiment, the cationic lipid is DOTMA and the additional lipid is DOPE.

In some embodiments, the molar ratio of the at least one cationic lipid to the at least one additional lipid is from about 10:0 to about 1:9, about 4:1 to about 1:2, or about 3:1 to about 1:1. In specific embodiments, the molar ratio may be about 3:1, about 2.75:1, about 2.5:1, about 2.25:1, about 2:1, about 1.75:1, about 1.5:1, about 1.25:1, or about 1:1. In an exemplary embodiment, the molar ratio of the at least one cationic lipid to the at least one additional lipid is about 2:1.

RNA lipoplex particles described herein have an average diameter that in one embodiment ranges from about 200 nm to about 1000 nm, from about 200 nm to about 800 nm, from about 250 to about 700 nm, from about 400 to about 600 nm, from about 300 nm to about 500 nm, or from about 350 nm to about 400 nm. In specific embodiments, the RNA lipoplex particles have an average diameter of about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 700 nm, about 725 nm, about 750 nm, about 775 nm, about 800 nm, about 825 nm, about 850 nm, about 875 nm, about 900 nm, about 925 nm, about 950 nm, about 975 nm, or about 1000 nm. In an embodiment, the RNA lipoplex particles have an average diameter that ranges from about 250 nm to about 700 nm. In another embodiment, the RNA lipoplex particles have an average diameter that ranges from about 300 nm to about 500 nm. In an exemplary embodiment, the RNA lipoplex particles have an average diameter of about 400 nm.

The electric charge of the RNA lipoplex particles of the present disclosure is the sum of the electric charges present in the at least one cationic lipid and the electric charges present in the RNA. The charge ratio is the ratio of the positive charges present in the at least one cationic lipid to the negative charges present in the RNA. The charge ratio of the positive charges present in the at least one cationic lipid to the negative charges present in the RNA is calculated by the following equation: charge ratio=[(cationic lipid concentration (mol))*(the total number of positive charges in the cationic lipid)]/[(RNA concentration (mol))*(the total number of negative charges in RNA)].

The spleen targeting RNA lipoplex particles described herein at physiological pH preferably have a net negative charge such as a charge ratio of positive charges to negative charges from about 1.9:2 to about 1:2. In specific embodiments, the charge ratio of positive charges to negative charges in the RNA lipoplex particles at physiological pH is about 1.9:2.0, about 1.8:2.0, about 1.7:2.0, about 1.6:2.0, about 1.5:2.0, about 1.4:2.0, about 1.3:2.0, about 1.2:2.0, about 1.1:2.0, or about 1:2.0.

Cytokines such as extended-PK cytokines, in particular extended-PK interleukins, such as those described herein may be provided to a subject by administering to the subject RNA encoding a cytokine in a formulation for preferential delivery of RNA to liver or liver tissue. The delivery of RNA to such target organ or tissue is preferred, in particular, if it is desired to express large amounts of the cytokine and/or if systemic presence of the cytokine, in particular in significant amounts, is desired or required.

RNA delivery systems have an inherent preference to the liver. This pertains to lipid-based particles, cationic and neutral nanoparticles, in particular lipid nanoparticles such as liposomes, nanomicelles and lipophilic ligands in bioconjugates. Liver accumulation is caused by the discontinuous nature of the hepatic vasculature or the lipid metabolism (liposomes and lipid or cholesterol conjugates).

For in vivo delivery of RNA to the liver, a drug delivery system may be used to transport the RNA into the liver by preventing its degradation. For example, polyplex nanomicelles consisting of a poly(ethylene glycol) (PEG)-coated surface and an mRNA-containing core is a useful system because the nanomicelles provide excellent in vivo stability of the RNA, under physiological conditions. Furthermore, the stealth property provided by the polyplex nanomicelle surface, composed of dense PEG palisades, effectively evades host immune defenses.

Pharmaceutical Compositions

The agents described herein may be administered in pharmaceutical compositions or medicaments and may be administered in the form of any suitable pharmaceutical composition.

In one embodiment of all aspects of the invention, the components described herein such as nucleic acid encoding a cytokine (IL2 or IFN) or nucleic acid encoding an antigen either together or separate from each other may be administered in a pharmaceutical composition which may comprise a pharmaceutically acceptable carrier and may optionally comprise one or more adjuvants, stabilizers etc. In one embodiment, the pharmaceutical composition is for therapeutic or prophylactic treatments, e.g., for use in treating or preventing a disease involving an antigen such as a cancer disease such as those described herein.

The term “pharmaceutical composition” relates to a formulation comprising a therapeutically effective agent, preferably together with pharmaceutically acceptable carriers, diluents and/or excipients. Said pharmaceutical composition is useful for treating, preventing, or reducing the severity of a disease or disorder by administration of said pharmaceutical composition to a subject. A pharmaceutical composition is also known in the art as a pharmaceutical formulation.

The pharmaceutical compositions of the present disclosure may comprise one or more adjuvants or may be administered with one or more adjuvants. The term “adjuvant” relates to a compound which prolongs, enhances or accelerates an immune response. Adjuvants comprise a heterogeneous group of compounds such as oil emulsions (e.g., Freund's adjuvants), mineral compounds (such as alum), bacterial products (such as Bordetella pertussis toxin), or immune-stimulating complexes. Examples of adjuvants include, without limitation, LPS, GP96, CpG oligodeoxynucleotides, growth factors, and cytokines, such as monokines, lymphokines, interleukins, chemokines. The cytokines may be IL1, IL2, IL3, IL4, IL5, IL6, IL7, IL8, IL9, I-10, IL12, IFNα, IFNγ, GM-CSF, LT-a. Further known adjuvants are aluminium hydroxide, Freund's adjuvant or oil such as Montanide® ISA51. Other suitable adjuvants for use in the present disclosure include lipopeptides, such as Pam3Cys.

The pharmaceutical compositions according to the present disclosure are generally applied in a “pharmaceutically effective amount” and in “a pharmaceutically acceptable preparation”.

The term “pharmaceutically acceptable” refers to the non-toxicity of a material which does not interact with the action of the active component of the pharmaceutical composition.

The term “pharmaceutically effective amount” or “therapeutically effective amount” refers to the amount which achieves a desired reaction or a desired effect alone or together with further doses. In the case of the treatment of a particular disease, the desired reaction preferably relates to inhibition of the course of the disease. This comprises slowing down the progress of the disease and, in particular, interrupting or reversing the progress of the disease. The desired reaction in a treatment of a disease may also be delay of the onset or a prevention of the onset of said disease or said condition. An effective amount of the compositions described herein will depend on the condition to be treated, the severeness of the disease, the individual parameters of the patient, including age, physiological condition, size and weight, the duration of treatment, the type of an accompanying therapy (if present), the specific route of administration and similar factors. Accordingly, the doses administered of the compositions described herein may depend on various of such parameters. In the case that a reaction in a patient is insufficient with an initial dose, higher doses (or effectively higher doses achieved by a different, more localized route of administration) may be used.

The pharmaceutical compositions of the present disclosure may contain salts, buffers, preservatives, and optionally other therapeutic agents. In one embodiment, the pharmaceutical compositions of the present disclosure comprise one or more pharmaceutically acceptable carriers, diluents and/or excipients.

Suitable preservatives for use in the pharmaceutical compositions of the present disclosure include, without limitation, benzalkonium chloride, chlorobutanol, paraben and thimerosal.

The term “excipient” as used herein refers to a substance which may be present in a pharmaceutical composition of the present disclosure but is not an active ingredient. Examples of excipients, include without limitation, carriers, binders, diluents, lubricants, thickeners, surface active agents, preservatives, stabilizers, emulsifiers, buffers, flavoring agents, or colorants.

The term “diluent” relates a diluting and/or thinning agent. Moreover, the term “diluent” includes any one or more of fluid, liquid or solid suspension and/or mixing media. Examples of suitable diluents include ethanol, glycerol and water.

The term “carrier” refers to a component which may be natural, synthetic, organic, inorganic in which the active component is combined in order to facilitate, enhance or enable administration of the pharmaceutical composition. A carrier as used herein may be one or more compatible solid or liquid fillers, diluents or encapsulating substances, which are suitable for administration to subject. Suitable carrier include, without limitation, sterile water, Ringer, Ringer lactate, sterile sodium chloride solution, isotonic saline, polyalkylene glycols, hydrogenated naphthalenes and, in particular, biocompatible lactide polymers, lactide/glycolide copolymers or polyoxyethylene/polyoxy-propylene copolymers. In one embodiment, the pharmaceutical composition of the present disclosure includes isotonic saline.

Pharmaceutically acceptable carriers, excipients or diluents for therapeutic use are well known in the pharmaceutical art, and are described, for example, in Remington's Pharmaceutical Sciences, Mack Publishing Co. (A. R Gennaro edit. 1985).

Pharmaceutical carriers, excipients or diluents can be selected with regard to the intended route of administration and standard pharmaceutical practice.

In one embodiment, pharmaceutical compositions described herein may be administered intravenously, intraarterially, subcutaneously, intradermally or intramuscularly. In certain embodiments, the pharmaceutical composition is formulated for local administration or systemic administration. Systemic administration may include enteral administration, which involves absorption through the gastrointestinal tract, or parenteral administration. As used herein, “parenteral administration” refers to the administration in any manner other than through the gastrointestinal tract, such as by intravenous injection. In a preferred embodiment, the pharmaceutical compositions is formulated for systemic administration. In another preferred embodiment, the systemic administration is by intravenous administration. In one embodiment of all aspects of the invention, RNA encoding a cytokine described herein and optionally RNA encoding an antigen is administered systemically.

The term “co-administering” as used herein means a process whereby different compounds or compositions (e.g., RNA encoding a IL2 polypeptide, RNA encoding an IFN polypeptide, and optionally RNA encoding a vaccine antigen) are administered to the same patient. The different compounds or compositions may be administered simultaneously, at essentially the same time, or sequentially. In one embodiment, IL2 polypeptide or nucleic acid encoding IL2 polypeptide is administered first, followed by administration of IFN polypeptide or nucleic acid encoding IFN polypeptide.

Treatments

The agents, compositions and methods described herein can be used to treat a subject with a disease, e.g., a disease characterized by the presence of diseased cells expressing an antigen. Particularly preferred diseases are cancer diseases. For example, if the antigen is derived from a virus, the agents, compositions and methods may be useful in the treatment of a viral disease caused by said virus. If the antigen is a tumor antigen, the agents, compositions and methods may be useful in the treatment of a cancer disease wherein cancer cells express said tumor antigen.

The term “disease” refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune diseases. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality.

In the present context, the term “treatment”, “treating” or “therapeutic intervention” relates to the management and care of a subject for the purpose of combating a condition such as a disease or disorder. The term is intended to include the full spectrum of treatments for a given condition from which the subject is suffering, such as administration of the therapeutically effective compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of an individual for the purpose of combating the disease, condition or disorder and includes the administration of the active compounds to prevent the onset of the symptoms or complications.

The term “therapeutic treatment” relates to any treatment which improves the health status and/or prolongs (increases) the lifespan of an individual. Said treatment may eliminate the disease in an individual, arrest or slow the development of a disease in an individual, inhibit or slow the development of a disease in an individual, decrease the frequency or severity of symptoms in an individual, and/or decrease the recurrence in an individual who currently has or who previously has had a disease.

The terms “prophylactic treatment” or “preventive treatment” relate to any treatment that is intended to prevent a disease from occurring in an individual. The terms “prophylactic treatment” or “preventive treatment” are used herein interchangeably.

The terms “individual” and “subject” are used herein interchangeably. They refer to a human or another mammal (e.g. mouse, rat, rabbit, dog, cat, cattle, swine, sheep, horse or primate) that can be afflicted with or is susceptible to a disease or disorder (e.g., cancer) but may or may not have the disease or disorder. In many embodiments, the individual is a human being. Unless otherwise stated, the terms “individual” and “subject” do not denote a particular age, and thus encompass adults, elderlies, children, and newborns. In embodiments of the present disclosure, the “individual” or “subject” is a “patient”.

The term “patient” means an individual or subject for treatment, in particular a diseased individual or subject.

In one embodiment of the disclosure, the aim is to provide an immune response against diseased cells expressing an antigen such as cancer cells expressing a tumor antigen, and to treat a disease such as a cancer disease involving cells expressing an antigen such as a tumor antigen.

As used herein, “immune response” refers to an integrated bodily response to an antigen or a cell expressing an antigen and refers to a cellular immune response and/or a humoral immune response. “Cell-mediated immunity”, “cellular immunity”, “cellular immune response”, or similar terms are meant to include a cellular response directed to cells characterized by expression of an antigen, in particular characterized by presentation of an antigen with class I or class II MHC. The cellular response relates to cells called T cells or T lymphocytes which act as either “helpers” or “killers”. The helper T cells (also termed CD4⁺ T cells) play a central role by regulating the immune response and the killer cells (also termed cytotoxic T cells, cytolytic T cells, CD8⁺ T cells or CTLs) kill diseased cells such as cancer cells, preventing the production of more diseased cells.

The present disclosure contemplates an immune response that may be protective, preventive, prophylactic and/or therapeutic. As used herein, “induces [or inducing] an immune response” may indicate that no immune response against a particular antigen was present before induction or it may indicate that there was a basal level of immune response against a particular antigen before induction, which was enhanced after induction. Therefore, “induces [or inducing] an immune response” includes “enhances [or enhancing] an immune response”.

The term “immunotherapy” relates to the treatment of a disease or condition by inducing, or enhancing an immune response. The term “immunotherapy” includes antigen immunization or antigen vaccination.

The terms “immunization” or “vaccination” describe the process of administering an antigen to an individual with the purpose of inducing an immune response, for example, for therapeutic or prophylactic reasons.

The term “macrophage” refers to a subgroup of phagocytic cells produced by the differentiation of monocytes. Macrophages which are activated by inflammation, immune cytokines or microbial products nonspecifically engulf and kill foreign pathogens within the macrophage by hydrolytic and oxidative attack resulting in degradation of the pathogen. Peptides from degraded proteins are displayed on the macrophage cell surface where they can be recognized by T cells, and they can directly interact with antibodies on the B cell surface, resulting in T and B cell activation and further stimulation of the immune response. Macrophages belong to the class of antigen presenting cells. In one embodiment, the macrophages are splenic macrophages.

The term “dendritic ceil” (DC) refers to another subtype of phagocytic cells belonging to the class of antigen presenting cells. In one embodiment, dendritic cells are derived from hematopoietic bone marrow progenitor cells. These progenitor cells initially transform into immature dendritic cells. These immature cells are characterized by high phagocytic activity and low T cell activation potential. Immature dendritic cells constantly sample the surrounding environment for pathogens such as viruses and bacteria. Once they have come into contact with a presentable antigen, they become activated into mature dendritic cells and begin to migrate to the spleen or to the lymph node. Immature dendritic cells phagocytose pathogens and degrade their proteins into small pieces and upon maturation present those fragments at their cell surface using MHC molecules. Simultaneously, they upregulate cell-surface receptors that act as co-receptors in T cell activation such as CD80, CD86, and CD40 greatly enhancing their ability to activate T cells. They also upregulate CCR7, a chemotactic receptor that induces the dendritic cell to travel through the blood stream to the spleen or through the lymphatic system to a lymph node. Here they act as antigen-presenting cells and activate helper T cells and killer T cells as well as B cells by presenting them antigens, alongside non-antigen specific co-stimulatory signals. Thus, dendritic cells can actively induce a T cell- or B cell-related immune response. In one embodiment, the dendritic cells are splenic dendritic cells.

The term “antigen presenting cell” (APC) is a cell of a variety of cells capable of displaying, acquiring, and/or presenting at least one antigen or antigenic fragment on (or at) its cell surface. Antigen-presenting cells can be distinguished in professional antigen presenting cells and non-professional antigen presenting cells.

The term “professional antigen presenting cells” relates to antigen presenting cells which constitutively express the Major Histocompatibility Complex class II (MHC class II) molecules required for interaction with naïve T cells. If a T cell interacts with the MHC class II molecule complex on the membrane of the antigen presenting cell, the antigen presenting cell produces a co-stimulatory molecule inducing activation of the T cell. Professional antigen presenting cells comprise dendritic cells and macrophages.

The term “non-professional antigen presenting cells” relates to antigen presenting cells which do not constitutively express MHC class II molecules, but upon stimulation by certain cytokines such as interferon-gamma. Exemplary, non-professional antigen presenting cells include fibroblasts, thymic epithelial cells, thyroid epithelial cells, glial cells, pancreatic beta cells or vascular endothelial cells.

“Antigen processing” refers to the degradation of an antigen into procession products, which are fragments of said antigen (e.g., the degradation of a protein into peptides) and the association of one or more of these fragments (e.g., via binding) with MHC molecules for presentation by cells, such as antigen presenting cells to specific T cells.

The term “disease involving an antigen” refers to any disease which implicates an antigen, e.g. a disease which is characterized by the presence of an antigen. The disease involving an antigen can be an infectious disease, or a cancer disease or simply cancer. As mentioned above, the antigen may be a disease-associated antigen, such as a tumor-associated antigen, a viral antigen, or a bacterial antigen. In one embodiment, a disease involving an antigen is a disease involving cells expressing an antigen, preferably on the cell surface.

The term “infectious disease” refers to any disease which can be transmitted from individual to individual or from organism to organism, and is caused by amicrobial agent (e.g. common cold). Infectious diseases are known in the art and include, for example, a viral disease, a bacterial disease, or a parasitic disease, which diseases are caused by a virus, a bacterium, and a parasite, respectively. In this regard, the infectious disease can be, for example, hepatitis, sexually transmitted diseases (e.g. chlamydia or gonorrhea), tuberculosis, HIV/acquired immune deficiency syndrome (AIDS), diphtheria, hepatitis B, hepatitis C, cholera, severe acute respiratory syndrome (SARS), the bird flu, and influenza.

The terms “cancer disease” or “cancer” refer to or describe the physiological condition in an individual that is typically characterized by unregulated cell growth. Examples of cancers include, but are not limited to, carcinoma, lymphoma, blastoma, sarcoma, and leukemia. More particularly, examples of such cancers include bone cancer, blood cancer lung cancer, liver cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” according to the disclosure also comprises cancer metastases.

Combination strategies in cancer treatment may be desirable due to a resulting synergistic effect, which may be considerably stronger than the impact of a monotherapeutic approach. In one embodiment, the pharmaceutical composition is administered with an immunotherapeutic agent. As used herein “immunotherapeutic agent” relates to any agent that may be involved in activating a specific immune response and/or immune effector function(s). The present disclosure contemplates the use of an antibody as an immunotherapeutic agent. Without wishing to be bound by theory, antibodies are capable of achieving a therapeutic effect against cancer cells through various mechanisms, including inducing apoptosis, block components of signal transduction pathways or inhibiting proliferation of tumor cells. In certain embodiments, the antibody is a monoclonal antibody. A monoclonal antibody may induce cell death via antibody-dependent cell mediated cytotoxicity (ADCC), or bind complement proteins, leading to direct cell toxicity, known as complement dependent cytotoxicity (CDC). Non-limiting examples of anti-cancer antibodies and potential antibody targets (in brackets) which may be used in combination with the present disclosure include: Abagovomab (CA-125), Abciximab (CD41), Adecatumumab (EpCAM), Afutuzumab (CD20), Alacizumab pegol (VEGFR2), Altumomab pentetate (CEA), Amatuximab (MORAb-009), Anatumomab mafenatox (TAG-72), Apolizumab (HLA-DR), Arcitumomab (CEA), Atezolizumab (PD-L1), Bavituximab (phosphatidylserine), Bectumomab (CD22), Belimumab (BAFF), Bevacizumab (VEGF-A), Bivatuzumab mertansine (CD44 v6), Blinatumomab (CD 19), Brentuximab vedotin (CD30 TNFRSF8), Cantuzumab mertansin (mucin CanAg), Cantuzumab ravtansine (MUC1), Capromab pendetide (prostatic carcinoma cells), Cariumab (CNT0888), Catumaxomab (EpCAM, CD3), Cetuximab (EGFR), Citatuzumab bogatox (EpCAM), Cixutumumab (IGF-1 receptor), Claudiximab (Claudin), Clivatuzumab tetraxetan (MUC1), Conatumumab (TRAIL-R2), Dacetuzumab (CD40), Dalotuzumab (insulin-like growth factor I receptor), Denosumab (RANKL), Detumomab (B-lymphoma cell), Drozitumab (DR5), Ecromeximab (GD3 ganglioside), Edrecolomab (EpCAM), Elotuzumab (SLAMF7), Enavatuzumab (PDL192), Ensituximab (NPC-1C), Epratuzumab (CD22), Ertumaxomab (HER2/neu, CD3), Etaracizumab (integrin αvβ3), Farletuzumab (folate receptor 1), FBTA05 (CD20), Ficlatuzumab (SCH 900105), Figitumumab (IGF-1 receptor), Flanvotumab (glycoprotein 75), Fresolimumab (TGF-β), Galiximab (CD80), Ganitumab (IGF-1), Gemtuzumab ozogamicin (CD33), Gevokizumab (ILIβ), Girentuximab (carbonic anhydrase 9 (CA-IX)), Glembatumumab vedotin (GPNMB), Ibritumomab tiuxetan (CD20), Icrucumab (VEGFR-1), Igovoma (CA-125), Indatuximab ravtansine (SDC1), Intetumumab (CD51), Inotuzumab ozogamicin (CD22), Ipilimumab (CD 152), Iratumumab (CD30), Labetuzumab (CEA), Lexatumumab (TRAIL-R2), Libivirumab (hepatitis B surface antigen), Untuzumab (CD33), Lorvotuzumab mertansine (CD56), Lucatumumab (CD40), Lumiliximab (CD23), Mapatumumab (TRAIL-R1), Matuzumab (EGFR), Mepolizumab (IL5), Milatuzumab (CD74), Mitumomab (GD3 ganglioside), Mogamulizumab (CCR4), Moxetumomab pasudotox (CD22), Nacolomab tafenatox (C242 antigen), Naptumomab estafenatox (5T4), Namatumab (RON), Necitumumab (EGFR), Nimotuzumab (EGFR), Nivolumab (IgG4), Ofatumumab (CD20), Olaratumab (PDGF-R a), Onartuzumab (human scatter factor receptor kinase), Oportuzumab monatox (EpCAM), Oregovomab (CA-125), Oxelumab (OX-40), Panitumumab (EGFR), Patritumab (HER3), Pemtumoma (MUC1), Pertuzuma (HER2/neu), Pintumomab (adenocarcinoma antigen), Pritumumab (vimentin), Racotumomab (N-glycolylneuraminic acid), Radretumab (fibronectin extra domain-B), Rafivirumab (rabies virus glycoprotein), Ramucirumab (VEGFR2), Rilotumumab (HGF), Rituximab (CD20), Robatumumab (IGF-1 receptor), Samalizumab (CD200), Sibrotuzumab (FAP), Siltuximab (IL6), Tabalumab (BAFF), Tacatuzumab tetraxetan (alpha-fetoprotein), Taplitumomab paptox (CD 19), Tenatumomab (tenascin C), Teprotumumab (CD221), Ticilimumab (CTLA-4), Tigatuzumab (TRAIL-R2), TNX-650 (IL13), Tositumomab (CD20), Trastuzumab (HER2/neu), TRBS07 (GD2), Tremelimumab (CTLA-4), Tucotuzumab celmoleukin (EpCAM), Ublituximab (MS4A1), Urelumab (4-1 BB), Volociximab (integrin α5β1), Votumumab (tumor antigen CTAA 16.88), Zalutumumab (EGFR), and Zanolimumab (CD4).

Citation of documents and studies referenced herein is not intended as an admission that any of the foregoing is pertinent prior art. All statements as to the contents of these documents are based on the information available to the applicants and do not constitute any admission as to the correctness of the contents of these documents.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### EXAMPLES

### Example 1: Construct Design and mRNA Production

In vitro transcription of cytokine encoding mRNAs were based on the pST1-T7-AGA-dEarl-hAg-MCS-FI-A30LA70 plasmid-backbone and derivative DNA-constructs. These plasmid constructs contain a 5′ UTR (untranslated region, a derivate of the 5′-UTR of Homo sapiens hemoglobin subunit alpha 1 (hAg)), a 3′ FI element (where F is a 136 nucleotide long 3′-UTR fragment of amino-terminal enhancer of split mRNA and I is a 142 nucleotide long fragment of mitochondrially encoded 12S RNA both identified in Homo sapiens; WO 2017/060314) and a poly(A) tail of 100 nucleotides, with a linker after 70 nucleotides. Cytokine and serum albumin (Alb) encoding sequences originate from Mus musculus (abbreviated with m, i.e. mAlb, mIL2 or mIFNα) or Homo sapiens (abbreviated with h, i.e. hAlb, hIL2 hIFNα) no changes in the resulting amino acid sequences were introduced. Alb was introduced at the N-Terminus of the mature IL2 sequence (no signal peptide of IL2 was encoded). A stop-codon was introduced for the most C-terminal moiety only. Different protein moieties in the cytokine and hAlb fusion constructs were separated by a 30-nucleotide long linker sequence encoding for glycine and serine residues. mRNA was generated by in vitro transcription as described by Kreiter et al. (Kreiter, S. et al. Cancer Immunol. Immunother. 56, 1577-87 (2007)). For all Cytokine/Albumin encoding RNAs the normal nucleoside uridine was substituted by 1-methyl-pseudouridine. Resulting mRNAs were equipped with a Cap1-structure and double-stranded (dsRNA) molecules were depleted. Purified Cytokine/Albumin mRNA was eluted in H₂O and stored at −80° C. until further use. In vitro transcription of all described mRNA constructs was carried out at BioNTech RNA Pharmaceuticals GmbH. A list of all constructs used in subsequent experiments is shown in Table 1.

TABLE 1
Amino acid sequences of mRNA encoded and expressed proteins.

| | |
|---|---|
| mAlb | MKWVTFLLLLFVSGSAFSRGVFRREAHKSEIAHRYNDLGEQHFKGLVLIAFSQYLQKCSYD |
| | EHAKLVQEVTDFAKTCVADESAANCDKSLHTLFGDKLCAIPNLRENYGELADCCTKQEPE |
| | RNECFLQHKDDNPSLPPFERPEAEAMCTSFKENPTTFMGHYLHEVARRHPYFYAPELLyy |
| | AEQYNEILTQCCAEADKESCLTPKLDGVKEKALVSSVRQRMKCSSMQKFGERAFKAWAV |
| | ARLSQTFPNADFAEITKLATDLTKVNKECCHGDLLECADDRAELAOMCENQATISSKLQT |
| | CCDKPLLKKAHCLSEVEHDTMPADLPAIAADFVEDQEVCKNYAEAKDVFLGTFLYEYSRR |
| | HPDYSVSLLLRLAKKYEATLEKCCAEANPPACYGTVLAEFQPLVEEPKNLVKTNCDLYEKL |
| | GEYGFQNAILVRYTQKAPQVSTPTLVEAARNLGRVGTKCCTLPEDQRLPCVEDYLSAILNR |
| | VCLLHEKTPVSEHVTKCCSGSLVERRPCFSALTVDETYVPKEFKAETFTFHSDICTLPEKE |
| | KQIKKQTALAELVKHKPKATAEQLKTVMDDFAQFLDTCCKAADKDTCFSTEGPNLVTRCK |
| | DALA |
| mAlb-mIL2 | MKWVTFLLLLFVSGSAFSRGVFRREAHKSEIAHRYNDLGEQHFKGLVLIAFSQYLQKCSYD |
| | EHAKLVQEVTDFAKTCVADESAANCDKSLHTLFGDKLCAIPNLRENYGELADCCTKQEPE |
| | RNECFLQHKDDNPSLPPFERPEAEAMCTSFKENPTTFMGHYLHEVARRHPYFYAPELLYY |
| | AEQYNEILTQCCAEADKESCLTPKLDGVKEKALVSSVRQRMKCSSMQKFGERAFKAWAV |
| | ARLSQTFPNADFAEITKLATDLTKVNKECCHGDLLECADDRAELAKYMCENQATISSKLQT |
| | CCDKPLLKKAHCLSEVEHDTMPADLPAIAADFVEDQEVCKNYAEAKDVFLGTFLyEYSRR |
| | HPDYSVSLLLRLAKKYEATLEKCCAEANPPACYGTVLAEFQPLVEEPKNLVKTNCDLYEKL |
| | GEYGFQNAILVRYTQMPQVSTPTLVEAARNLGRVGTKCCTLPEDQRLPCVEDYLSAILNR |
| | VCLLHEKTPVSEHVTKCCSGSLVERRPCFSALTVDETYVPKEFKAETFTFHSDICTLPEKE |
| | KQIKKQTALAELVKHKPKATAEQLKTVMDDFAQFLDTCCKAADKDTCFSTEGPNLVTRCK |
| | DALAGGSGGGGSGGAPTSSSTSSSTAEAQQQQQQQQQQQQHLEQLLMDLQELLSRME |
| | NYRNLKLPRMLTFKFYLPKQATELKDLQCLEDELGPLRHVLDLTQSKSFQLEDAENFISNIR |
| | VTVVKLKGSDNTFECQFDDESATVVDFLRRWIAFCQSIISTSPQ |
| mIFNa | MGAMAPRILLLLLAAALAPTQTRAGPGSCDLPHTYNLGNKRALTVLEEMRRLIDPLSCLKD |
| | RKDFGFPLEKVDNQQIQKAQAILVLRDLTQQILNLFTSKDLSATWNATLLDSFCNDLHQQL |
| | NDLKACVMQEPPLTQEDSLLAVRTYFHRITVYLRKKKHSLCAWEVIRAEVWRALSSSTNLL |
| | ARLSEEKE |
| hAlb | MKWVTFISLLFLFSSAYSRGVFRRDAHKSEVAHRFKDLGEENFKALVLIAFAQYLQQCPF |
| | EDHVKLVNEVTEFAKTCVADESAENCDKSLHTLFGDKLCTVATLRETYGEMADCCAKQEP |
| | ERNECFLQHKDDNPNLPRLVRPEVDVMCTAFHDNEETFLKKYLYEIARRHPYFYAPELLF |
| | FAKRYAAFTECCQAADKAACLLPKLDELRDEGKASSAKQRLKCASLQKFGERAFKAWAV |
| | ARLSQRFPMEFAEVSKLVTDLTKVHTECCHGDLLECADDRADLAKYICENQDSISSKLK |
| | ECCEKPLLEKSHCIAEVENDEMPADLPSLAADFVESKDVCKNYAEAKDVFLGMFLYEYAR |
| | RHPDYSVVLLLRLAKTYETTLEKCCAAADPHECYAKVFDEFKPLVEEPQNLIKQNCELFE |
| | QLGEYKFQNALLVRYTKKVPQVSTPTLVEVSRNLGKVGSKCCKHPEAKRMPCAEDYLSVV |
| | LNQLCVLHEKTPVSDRVTKCCTESLVNRRPCFSALEVDETYVPKEFNAETFTFHADICTL |
| | SEKERQIKKQTALVELVKHKPKATKEQLKAVMDDFAAFVEKCCKADDKETCFAEEGKKLV |
| | AASQAALGL |
| hAlb-hIL2 | MKWVTFISLLFLFSSAYSRGVFRRDAHKSEVAHRFKDLGEENFKALVLIAFAQYLQQCPF |
| | EDHVKLVNEVTEFAKTCVADESAENCDKSLHTLFGDKLCTVATLRETYGEMADCCAKQEP |
| | ERNECFLQHKDDNPNLPRLVRPEVDVMCTAFHDNEETFLKKYLYEIARRHPYFYAPELLF |
| | FAKRYFTECCQAADKAACLLPKLDELRDEGKASSAKQRLKCASLQKFGERAFKAWAV |
| | ARLSQRFPKAEFAEVSKLVTDLTKVHTECCHGDLLECADDRADLAKYICENQDSISSKLK |
| | ECCEKPLLEKSHCIAEVENDEMPADLPSLAADFVESKDVCKNYAEAKDVFLGMFLYEyAR |
| | RHPDYSVVLLLRLAKTYETTLEKCCAAADPHECYAKVFDEFKPLVEEPQNLIKQNCELFE |
| | QLGEYKFQNALLVRYTKKVPQVSTPTLVEVSRNLGKVGSKCCKHPEAKRMPCAEDYLSVV |
| | LNQLCVLHEKTPVSDRVTKCCTESLVNRRPCFSALEVDETYVPKEFNAETFTFHADICTL |
| | SEKERQIKKQTALVELVKHKPKATKEQLKAVMDDFAAFVEKCCKADDKETCFAEEGKKLV |
| | AASQAALGLGGSGGGGSGGAPTSSSTKKTQLQLEHLLLDLQMILNGINNYKNPKLTRMLT |
| | FKFYMPKKATELKHLQCLEEELKPLEEVLNLAQSKNFHLRPRDLISNINVIVLELKGSET |
| | TFMCEYADETATIVEFLNRWITFCQSIISTLT |

### Example 2: Effects of RNA-Encoded IL2 on Vaccine Induced T-Cell Responses In Vivo

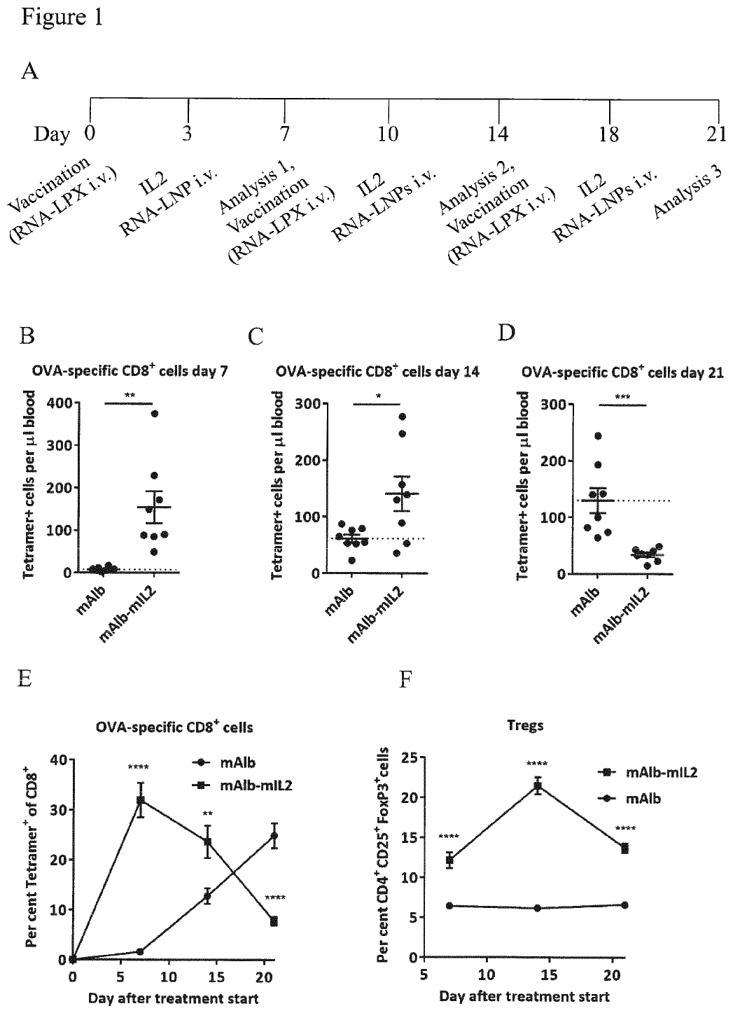

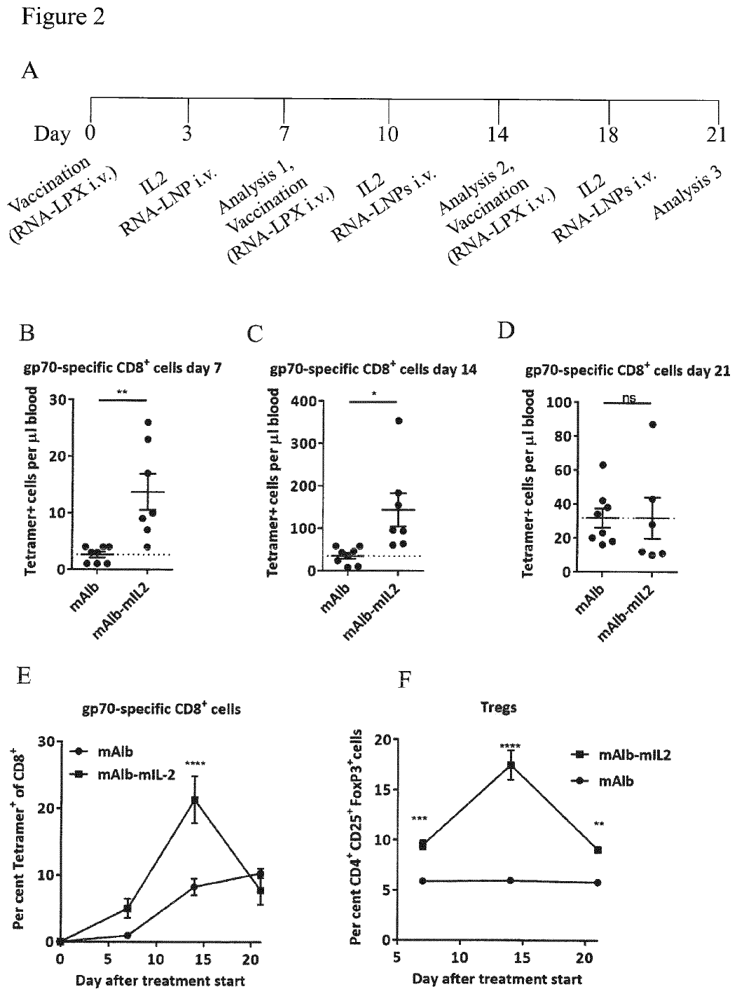

Female C57BL/6 (9 weeks old) (n=8 mice per group) were purchased from Envigo and vaccinated intravenously (i.v.) with 10 μg mRNA Lipoplexes (RNA-L) encoding the chicken ovalbumin (OVA) derived H2-Kb restricted CD8⁺ T-cell epitope SIINFEKL (Kranz, L. M. et al. Nature 534, 396-401 (2016)) on day 0, 7 and 14. Three days after each vaccination 1 μg mAlb-fusion protein-encoding mRNA formulated with TransIT (Mirrus) was administered i.v.. Mice received either mAlb or mIL2 fused to mAlb (mAlb-mIL2). On day 7, 14 and 21 blood of mice was retrieved and analyzed for antigen specific T cell responses as well as regulatory T cells (Tregs) via flow cytometry. 50 μl of blood was stained for 30 min at 2-8° C. with fluorochrome labeled antibodies or MHC Tetramers. Antigen specific T cells were detected via co-staining of CD45 (30-F11, BD) and CD8 (clone 5H10, BD) specific antibodies as well as MHC Tetramers bound to SIINFEKL peptide (MBL international). Tregs were identified by antibodies specific for CD45 (30-F11, BD), CD4 (clone RM4-5, Biolegend), CD25 (clone PC61, BD) and FoxP3 (clone FJK-16s, eBioscience) using the FoxP3 staining kit of eBioscience according to the manufacturers instructions. Blood was lysed using lysing solution (BD FACS™). In order to determine absolute cell numbers, cells were transferred into Trucount® tubes (BD). Flow cytometric data were acquired on a LSRFortessa flow cytometer (BD) and analyzed with FlowJo X software (Tree Star). Results were depicted and statistics analyzed using GraphPad Prism 7. FIG. 1A gives an overview of the treatment and analysis schedule. Combination of RNA-L vaccination and treatment with mAlb-mIL2 resulted in a significant increase of OVA-specific T cells on day 7 and 14 compared to the control group that only received RNA-L vaccination and mAlb (FIG. 1B, C). However, analysis on day 21 revealed a significant drop of antigens specific T-cell responses in mAlb-mIL2 treated animals with frequencies even significantly below the mAlb control (FIG. 1D, E). We hypothesized that this drop of antigen specific T cells was caused by mAlb-mIL2 induced Tregs. Indeed, mAlb-mIL2 treated mice showed significantly higher frequencies of Tregs at every time point measured and with a peak at day 14 (FIG. 1F). Presumably, at early time points the stimulatory potential of mAlb-mIL2 exceeds the inhibitory potential of Tregs. At later time points Treg numbers increase resulting in a stronger inhibitory signal on activated CD8+ T cells undermining the stimulatory potential of mAlb-mIL2. To confirm these results in a second model in a different background BALB/c mice (n=7-8 per group, female, 9 weeks, purchased from Janvier Labs) were treated with 20 μg gp70 RNA-L vaccination encoding the H2-Kd restricted CD8 T-cell antigen SPSYAYHQF (Kranz, L. M. et al. Nature 534, 396-401 (2016)) in combination with 1 μg mAlb-mIL2 or mAlb as described in FIG. 2A. Again, treatment with mAlb-mIL2 resulted in a significant but temporary increase of gp70 specific T cells only on days 7 and 14 (FIG. 2B, C). On day 21, antigen specific T-cell numbers returned to the level of the mAlb control (FIG. 2D, E). As shown before, Treg frequencies were significantly elevated at all measurements (FIG. 2F).

### Example 3: IFNα Limits IL2 Mediated Treg Expansion Resulting in Robust Priming of Antigen Specific T Cells In Vivo

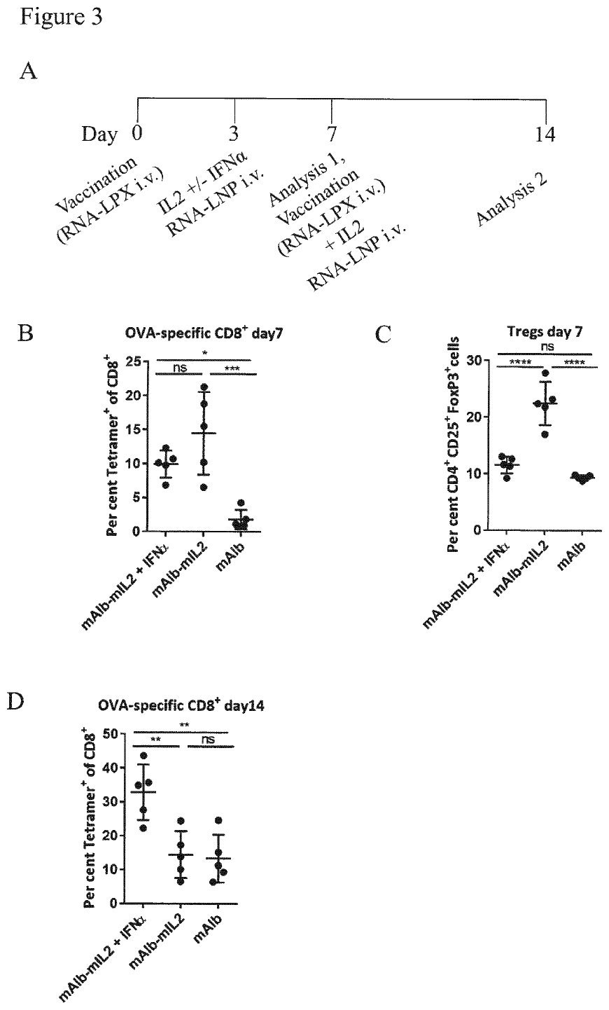

It has been demonstrated that IFNα administration can lead to a reduced frequency of Tregs in mice (Gangaplara, A. et al. PLoS Pathog. 14, 1-27 (2018).) as well as humans (Tatsugami, K., Eto, M. & Naito, S. J. Interferon Cytokine Res. 30, 43-48 (2010).) and can interfere with Treg function (Bacher, N. et al. Cancer Res. 73, 5647-5656 (2013).). However, IFNα also plays a positive role in Treg development (Metidji, A. et al. J. Immunol. 194, 4265-4276 (2015).). We hypothesized that IFNα might potentiate the effect of IL2 on vaccine induced T-cell priming by counteracting the immediate IL2 mediated Treg activation and expansion thereby preventing the subsequent inhibition of T-cell proliferation by Tregs. Therefore, we vaccinated C57BL/6 mice (9 weeks old, n=5 mice per group, purchased from Envigo) i.v. with 10 μg OVA RNA-L in 100 μl on day 0, 7 and 14 as described in Example 2. Three days after, mice were stimulated with 1 μg mAlb or mAlb-mIL2 encoding mRNA in 100 μl formulated with TransIT (Mirrus). Simultaneously, one mAlb-mIL2 treated group received 2 μg IFNα encoding mRNA in 100 μl formulated with TransIT (Mirrus). A second treatment with 10 μg OVA RNA-L and cytokine encoding RNA was performed on day 7 as shown in FIG. 3A. On day 7 and 14 blood of mice was retrieved and analyzed for antigen specific T-cell responses as well as Tregs via flow cytometry as described in Example 2. As expected, on day 7 vaccine induced OVA specific T-cells as well as Tregs were more frequent when mice were treated with mAlb-mIL2 (FIG. 3B, C). Co-administration of IFNα did not limit or increase the number of antigen specific T cells (FIG. 38) but reduced the number of Tregs to baseline levels of the mAb control (FIG. 3C). Consequently, analysis on day 14 showed that OVA specific T cells were significantly elevated in the group that received IFNα together with mAlb-mIL2 whereas sole treatment with mAlb-mIL2 had no benefit compared to the control group (FIG. 3D).

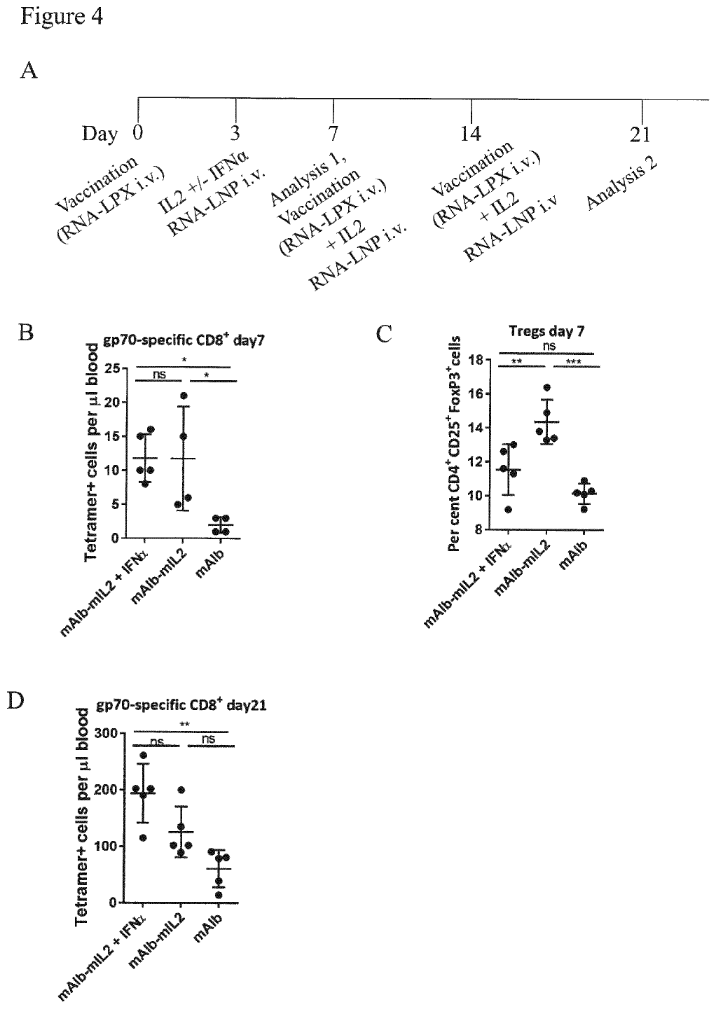

Confirmation of these results was conducted in the gp70 model described in Example 2 and FIG. 2. The experiment was performed similar as described above. BALB/c mice (n=5 per group) were vaccinated with 20 μg gp70 RNA-L in 100 μl on day 0, 7 and 14. Cytokine RNA (1 μg mAlb, 1 μg mAlb-mIL2 or 1 μg mAlb-mIL2 plus 2 μg IFNα) was injected in 100 μl on day 3, 7 as well as 14 and blood of mice was investigated on day 7 and 21 (FIG. 4A). Again, IFNα was able to normalize the mAlb-mIL2 mediated elevation of the Treg frequency without limiting the expansion of antigen specific T cells (FIG. 4 B, C) leading to an increase in antigen specific T cells on day 21 (FIG. 4D).

### Example 4: IFNα Limits IL2 Mediated Treg Expansion without Impairing CD8+ T Cell Expansion In Vitro

In order to support the in vivo observed beneficial effect of IFNα, the effect of hIFNa on IL2-stimulated CD4+CD25+ Tregs and CD8+ T cells as evaluated in vitro. Tregs and autologous bulk PBMCs were co-cultured at a 1:1 ratio in the presence of a sub-optimal concentration of anti-CD3 antibody (clone UCHT1) and 5% hAlb-hIL2-containing supernatant and additionally treated with IFNα or kept without IFNα. In short, human PBMCs were obtained from buffy coats of healthy donors by Ficoll-Paque (VWR International, cat no. 17-1440-03) density gradient separation and CD4+CD25+ Tregs were isolated from the freshly prepared PBMCs (CD4⁺CD25⁺ Regulatory T Cell Isolation Kit human, Miltenyi Biotec, cat. no. 130-091-301). Bulk PBMCs were labeled using 1.6 μM carboxyfluorescein succinimidyl ester (CFSE; Thermo Fisher, cat. no. C34554) and Tregs were labeled using 1 μM CellTrace FarRed dye (Thermo Fisher, cat. no. C34564). 30,000 CFSE-labeled PBMCs and 30,000 FarRed-labeled Tregs were co-cultured per well in a 96-well round-bottom plate (Costar, cat. no. 734-1797) in Iscove's Modified Dulbecco's Medium (IMDM; Life Technologies GmbH, cat. no. 12440-053) supplemented with 5% plasma-derived human serum (PHS; One Lambda Inc., cat. no. A25761) and incubated with a sub-optimal concentration of anti-CD3 antibody (UCHT1; R&D Systems, cat. no. MAB100; 0.09 μg/mL final concentration). PBMC:Treg co-cultures were treated with hAlb-hIL2-containing supernatants (5% final concentration) and either none, 625 U/mL or 10,000 U/mL recombinant IFNα2b (pbl assay science, cat. not. 11105-1) was added. Co-cultures were stimulated for four days at 37° C., 5% CO₂. Cells were harvested and analyzed by flow cytometry excluding dead cells via eFluor780 staining (eBioscience, cat. no. 65-0865-18). CD8+ T cells were identified by staining with anti-human CD8 PE-Cy7 antibody (TONBO Biosciences, cat. no. 60-0088) diluted 1:100 in FACS buffer (DPBS+2% FBS+2 mM EDTA). Flow cytometric analysis was performed on a BD FACS Canto II flow cytometer (Becton Dickinson) with CFSE-dilution (CD8+ T cells) and FarRed-dilution (Tregs) as proliferation read-out. Acquired proliferation data were analyzed using FlowJo 10.5 software (TreeStar, Inc.) and exported expansion index values were plotted in GraphPad Prism 6 (GraphPad Software, Inc.).

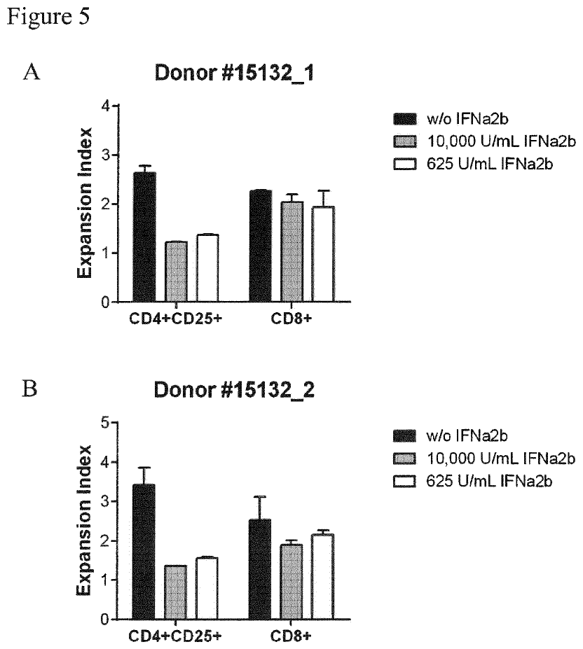

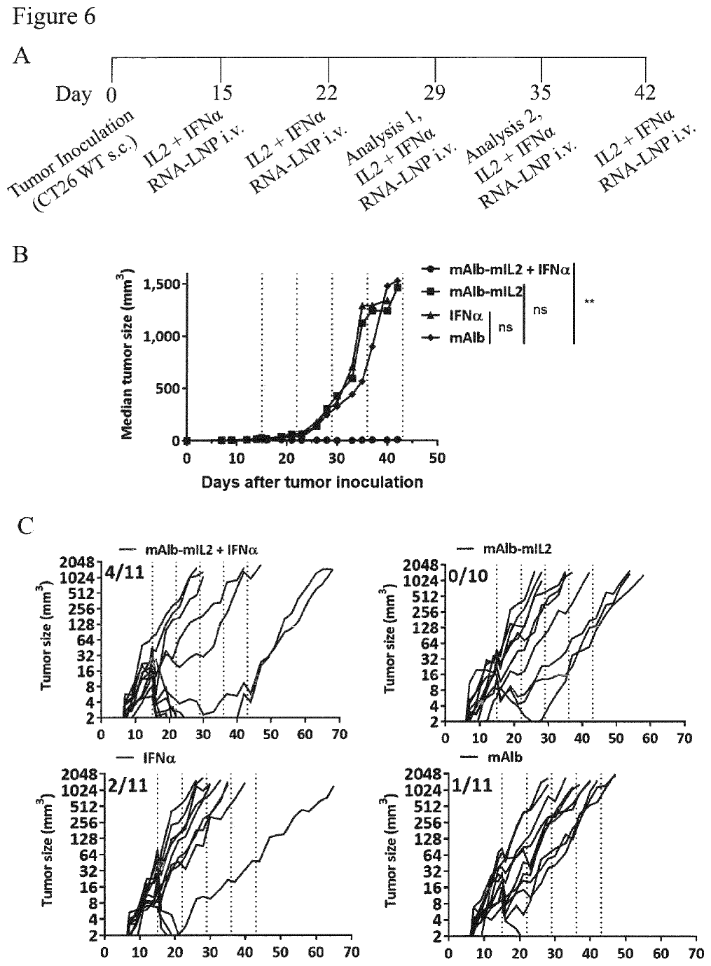

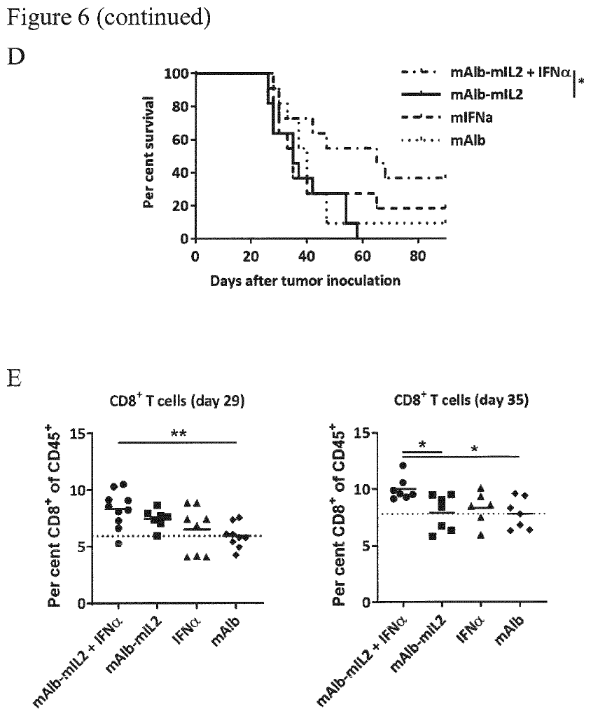

Both CD8+ T cells and CD4+CD25+ Tregs responded with strong proliferation upon combined anti-CD3 and hAlb-hIL2 treatment. While the addition of recombinant IFNα reduced the proliferation of Tregs by approximately 50-55%, the proliferation of CD8+ T cells was only decreased by approximately 10-20%. The effect of selective proliferation inhibition of Tregs but not CD8+ T cells was observed for both tested PBMC donors (FIG. 5 A, B) and largely independent of the used IFNα concentration.

### Example 5: IFNα and IL2 Combination Therapy Leads to a Synergistic Anti-Tumoral Effect in Mice

Next, we assessed whether addition of IFNα to IL2 therapy would result in an improved anti-tumor effect. Female BALB/c mice (9 weeks old, n=12 mice per group) were purchased from Janvier Labs S.A.S. and injected subcutaneously (s.c.) with 5×10⁵ CT26 colon carcinoma cells. 15 days after tumor cell inoculation, 10-11 mice per group were stratified based on tumor size. Mice were treated five times with 1 μg mAlb-mIL2 encoding RNA and 2 μg IFNα encoding mRNA separately formulated with TransIT (Mirrus) in 100 μL. Control groups received either IFNα, IL2 or an irrelevant RNA (1 μg mAlb encoding RNA) formulated with TransIT. Tumor sizes were measured with a caliper three time per week and calculated using the equation (a²×b)/2 (a, width; b, length). Animals were euthanized when exhibiting signs of impaired health or when the tumor volume exceeded 1500 mm³. On day 29 and 35, blood of mice was retrieved and CD8⁺ T cells were analyzed via flow cytometry as described in Example 2. FIG. 6 A illustrates the experimental outline.

Of note, no vaccine was added in this experiment since CT26 tumors are per se immunogenic and tumor specific T cells can be primed without a vaccine, particularly under IL2 treatment. Moreover, in murine tumors, especially early interventions determine the therapeutic outcome. The IL2 mediated boost of vaccine induced T cells would thus result in strong tumor control and the subsequent inhibition of T cells by IL2 stimulated Tregs would not carry weight. In contrast, without vaccination, the boosting effect of IL2 on tumor specific T cells is less pronounced and therefore the inhibitory effect of Tregs on tumor specific T cells more consequential.

Significant therapeutic activity was detected in the group treated with a combination of IFNα and IL2 whereas IL2 or IFNα monotherapy did not affect tumor growth (FIG. 6 B). In total, four out of 11 mice (36%) rejected their tumor under combination therapy whereas only two (18%) or none of the mice were tumor free following monotherapy with IFNα or IL2, respectively (FIG. 6 C). IL2 and IFNα treatment resulted in a significant survival benefit as compared to IL2 monotherapy (FIG. 6 D). Importantly, therapeutic activity of IL2 and IFNα combination therapy was accompanied by a consistent and significant increase of CD8⁺ T cells in the blood. Similar to FIG. 2 E and FIG. 3 D, IL2 monotherapy mediated only a transient elevation of CD8⁺ T cells at day 29 which normalized until day 35 (FIG. 6 E).

1. A method for inducing an immune response in a subject comprising administering to the subject:
a. a polypeptide comprising IL2 or a functional variant thereof or a polynucleotide encoding a polypeptide comprising IL2 or a functional variant thereof; and
b. a polypeptide comprising type I interferon or a functional variant thereof or a polynucleotide encoding a polypeptide comprising type I interferon or a functional variant thereof.

2. The method of claim 1 which further comprises administering to the subject:
c. a peptide or protein comprising an epitope for inducing an immune response against an antigen in the subject or a polynucleotide encoding the peptide or protein.

3. The method of claim 1 or 2, wherein the polynucleotide encoding a polypeptide comprising IL2 or a functional variant thereof is RNA, the polynucleotide encoding a polypeptide comprising type I interferon or a functional variant thereof is RNA and optionally the polynucleotide encoding the peptide or protein is RNA.

4. A method for inducing an immune response in a subject comprising administering to the subject:
a. RNA encoding a polypeptide comprising IL2 or a functional variant thereof; and
b. RNA encoding a polypeptide comprising type I interferon or a functional variant thereof.

5. The method of claim 4 which further comprises administering to the subject:
c. RNA encoding a peptide or protein comprising an epitope for inducing an immune response against an antigen in the subject.

6. The method of any one of claims 1 to 5, wherein the immune response is a T cell-mediated immune response.

7. The method of any one of claims 1 to 6, wherein the subject has a disease, disorder or condition associated with expression or elevated expression of an antigen.

8. A method for treating a subject having a disease, disorder or condition associated with expression or elevated expression of an antigen comprising administering to the subject:
a. a polypeptide comprising IL2 or a functional variant thereof or a polynucleotide encoding a polypeptide comprising IL2 or a functional variant thereof;
b. a polypeptide comprising type I interferon or a functional variant thereof or a polynucleotide encoding a polypeptide comprising type I interferon or a functional variant thereof; and
c. a peptide or protein comprising an epitope for inducing an immune response against the antigen in the subject or a polynucleotide encoding the peptide or protein.

9. The method of claim 8, wherein the polynucleotide encoding a polypeptide comprising IL2 or a functional variant thereof is RNA, the polynucleotide encoding a polypeptide comprising type I interferon or a functional variant thereof is RNA and the polynucleotide encoding the peptide or protein is RNA.

10. A method for treating a subject having a disease, disorder or condition associated with expression or elevated expression of an antigen comprising administering to the subject:
a. RNA encoding a polypeptide comprising IL2 or afunctional variant thereof;
b. RNA encoding a polypeptide comprising type I interferon or a functional variant thereof; and
c. RNA encoding a peptide or protein comprising an epitope for inducing an immune response against the antigen in the subject.

11. The method of any one of claims 7 to 10, wherein the disease, disorder or condition is cancer and the antigen is a tumor-associated antigen.

12. The method of any one of claims 1 to 11, wherein the polypeptide comprising IL2 or a functional variant thereof is extended pharmacokinetic (PK) IL2.

13. The method of claim 12, wherein the extended-PK IL2 comprises a fusion protein.

14. The method of claim 13, wherein the fusion protein comprises a moiety of IL2 or a functional variant thereof and a moiety selected from the group consisting of serum albumin, an immunoglobulin fragment, transferrin, Fn3, and variants thereof.

15. The method of claim 14, wherein the serum albumin comprises mouse serum albumin or human serum albumin.

16. The method of claim 14, wherein the immunoglobulin fragment comprises an immunoglobulin Fc domain.

17. The method of any one of claims 1 to 16, which is a method for treating or preventing cancer in a subject, optionally wherein the antigen is a tumor-associated antigen.

18. The method of any one of claims 1 to 17, wherein administration of a polypeptide comprising IL2 or a functional variant thereof or a polynucleotide, in particular RNA, encoding a polypeptide comprising IL2 or a functional variant thereof and optionally a peptide or protein comprising an epitope for inducing an immune response against an antigen in the subject or a polynucleotide, in particular RNA, encoding the peptide or protein, induces antigen-specific T cells.

19. The method of any one of claims 1 to 18, wherein administration of a polypeptide comprising type I interferon or a functional variant thereof or a polynucleotide, in particular RNA, encoding a polypeptide comprising type I interferon or a functional variant thereof reduces the number of regulatory T cells.

20. The method of any one of claims 1 to 19, wherein administration of a polypeptide comprising type I interferon or a functional variant thereof or a polynucleotide, in particular RNA, encoding a polypeptide comprising type I interferon or a functional variant thereof reduces IL2 mediated expansion of regulatory T cells.

21. The method of any one of claims 1 to 20, wherein administration of a polypeptide comprising type I interferon or a functional variant thereof or a polynucleotide, in particular RNA, encoding a polypeptide comprising type I interferon or a functional variant thereof increases the ratio of antigen-specific T cells to T regulatory cells.

22. The method of any one of claims 1 to 21, wherein the type I interferon is interferon-α.

23. A medical preparation comprising:
a. a polypeptide comprising IL2 or a functional variant thereof or a polynucleotide encoding a polypeptide comprising IL2 or afunctional variant thereof; and
b. a polypeptide comprising type I interferon or a functional variant thereof or a polynucleotide encoding a polypeptide comprising type I interferon or a functional variant thereof.

24. The medical preparation of claim 23 which further comprises:
c. a peptide or protein comprising an epitope for inducing an immune response against an antigen in a subject or a polynucleotide encoding the peptide or protein.

25. The medical preparation of claim 23 or 24, wherein the polynucleotide encoding a polypeptide comprising IL2 or a functional variant thereof is RNA, the polynucleotide encoding a polypeptide comprising type I interferon or a functional variant thereof is RNA and optionally the polynucleotide encoding the peptide or protein is RNA.

26. The medical preparation of any one of claims 23 to 25, which is a kit.

27. The medical preparation of claim 26, which comprises the polypeptide comprising IL2 or a functional variant thereof or polynucleotide encoding a polypeptide comprising IL2 or a functional variant thereof, the polypeptide comprising type I interferon or a functional variant thereof or polynucleotide encoding a polypeptide comprising type I interferon or afunctional variant thereof and optionally the peptide or protein or polynucleotide encoding the peptide or protein in separate containers.

28. The medical preparation of claim 26 or 27, further comprising instructions for use of the medical preparation for treating or preventing cancer, optionally wherein the antigen is a tumor-associated antigen.

29. The medical preparation of any one of claims 23 to 25, which is a pharmaceutical composition.

30. The medical preparation of claim 29, wherein the pharmaceutical composition further comprises one or more pharmaceutically acceptable carriers, diluents and/or excipients.

31. A medical preparation comprising:
a. RNA encoding a polypeptide comprising IL2 or afunctional variant thereof; and
b. RNA encoding a polypeptide comprising type I interferon or a functional variant thereof.

32. The medical preparation of claim 31 which further comprises:
c. RNA encoding a peptide or protein comprising an epitope for inducing an immune response against an antigen in a subject.

33. The medical preparation of claim 31 or 32, which is a kit.

34. The medical preparation of claim 33, which comprises the RNA encoding a polypeptide comprising IL2 or a functional variant thereof, the RNA encoding a polypeptide comprising type I interferon or a functional variant thereof and optionally the RNA encoding a peptide or protein in separate containers.

35. The medical preparation of claim 33 or 34, further comprising instructions for use of the medical preparation for treating or preventing cancer, optionally wherein the antigen is a tumor-associated antigen.

36. The medical preparation of claim 31 or 32, which is a pharmaceutical composition.

37. The medical preparation of claim 36, wherein the pharmaceutical composition further comprises one or more pharmaceutically acceptable carriers, diluents and/or excipients.

38. The medical preparation of any one of claims 24 to 30 and 32 to 37, wherein the immune response is a T cell-mediated immune response.

39. The medical preparation of any one of claims 23 to 38, wherein the polypeptide comprising IL2 or a functional variant thereof is extended pharmacokinetic (PK) IL2.

40. The medical preparation of claim 39, wherein the extended-PK IL2 comprises a fusion protein.

41. The medical preparation of claim 40, wherein the fusion protein comprises a moiety of IL2 or a functional variant thereof and a moiety selected from the group consisting of serum albumin, an immunoglobulin fragment, transferrin, Fn3, and variants thereof.

42. The medical preparation of claim 41, wherein the serum albumin comprises mouse serum albumin or human serum albumin.

43. The medical preparation of claim 41, wherein the immunoglobulin fragment comprises an immunoglobulin Fc domain.

44. The medical preparation of any one of claims 23 to 43 for pharmaceutical use.

45. The medical preparation of claim 44, wherein the pharmaceutical use comprises a therapeutic or prophylactic treatment of a disease or disorder.

46. The medical preparation of any one of claims 23 to 45 for use in a method for treating or preventing cancer in a subject, optionally wherein the antigen is a tumor-associated antigen.

47. The medical preparation of any one of claims 23 to 46, wherein the type I interferon is interferon-α.
